(19) United States
(12) Patent Application Publication
(10) Pub. No.: US 2014/0330376 A1
(43) Pub. Date: Nov. 6, 2014

(54) Apparatus And Method For The Treatment of Presbyopia in a Pseudophakic Eye

(71) Applicant: Cataract Innovations LLC, Rochester, NY (US)

(72) Inventor: David Maxwell Kleinman, Rochester, NY (US)

(73) Assignee: Cataract Innovations LLC, Rochester, NY (US)

(21) Appl. No.: 14/265,231

(22) Filed: Apr. 29, 2014

Related U.S. Application Data
(60) Provisional application No. 61/819,636, filed on May 5, 2013.

(51) Int. Cl. A61F 2/16

(52) U.S. Cl. CPC A61F 2/1648; A61F 2/1662
USPC 623/6.36; 606/107

(57) ABSTRACT

A method and apparatus to treat presbyopia or pseudophakic refractive error including but not limited to astigmatism and higher order aberrations is disclosed. An intraocular lens is pierced and the resulting opening becomes a pinhole device or becomes a dual lens system device to include a primary IOL and a secondary IOL for correction of error. The lens system device is secured to the in situ intraocular lens using surgical devices and methods of the present invention. The technique does not involve removal of the intraocular lens and replacement with another intraocular lens, but rather involves the addition of a lens system device that is secured to the intraocular lens in a stable manner.

### CROSS REFERENCE TO RELATED PATENT APPLICATIONS

This application claims priority to U.S. Patent Application Ser. No. 61/819,636 filed May 5, 2013 entitled “Apparatus And Method For The Treatment of Presbyopia in a Pseudophakic Eye” by Dr. David M. Kleinman. The disclosure of this U.S. Patent Application Ser. No. 61/819,636 is incorporated herein by reference in its entirety.

### BACKGROUND OF THE INVENTION

1. Field of the Invention

This invention relates generally to surgery of the eye, and more particularly to an apparatus and a method for the treatment of presbyopia in a pseudophakic patient.

2. Description of Related Art

Presbyopia is a condition in which the lens of the eye loses its ability to focus, making it difficult to see objects up close. The same process occurs after a patient is treated for cataract with the removal of the natural cloudy lens and placement of a single power, non-premium plastic intraocular lens (IOL). An IOL can be placed in the anterior chamber or posterior chamber of the eye. Current approaches to pseudophakic presbyopia include the placement of premium IOL's. A premium IOL addresses the near focus problem with either a multifocal lens or an accommodating lens. An accommodating lens changes shape or position to help focus at near. A multifocal IOL brings several different distances into focus at once. The standard for IOL surgery is to place a single power, non-premium IOL in the eye with the focus set for distance. Typically the accommodating and multifocal IOL's are also chosen based on their power for optimal distance acuity, and then through accommodation or multiple focal planes the near vision is improved. Another approach to presbyopia in pseudophakes includes monovision (where the eyes are set at different distances). An additional approach is corneal surgery where either a small intracorneal ring is placed to provide a pinhole effect centrally which can be used for phakic and pseudophakic patients, and a small power lens can be placed in the central intracorneal space as well. Presbyopia is a huge concern globally. Anyone older than approximately age 40 or 45 will develop presbyopia when fully corrected at distance. The problem of presbyopia is very common in patients who have undergone cataract surgery, especially those who received a single power IOL. Approximately 2.5 million Americans undergo cataract surgery annually and the majority receive a single vision power lens. There is a backlog of millions of people who have received a single vision IOL who now must use reading glasses for presbyopia.

Another major issue facing cataract patients involves the final postoperative refractive error. Generally the cataract surgeon selects a postoperative refraction close to plano correction at distance. Other times a patient may request a slightly myopic status. This choice can be based on the concern that a mild overcorrection could lead to hyperopia which is unsatisfactory because then refractive correction is required at all times, whereas myopes can see at some distance in focus. Thus, a first problem after cataract surgery is an unexpected hyperopic outcome. In this case, a lens exchange is considered. Another problem that can occur is that the wrong lens was placed, or ocular biometry predicted a much different result than was actually identified after cataract surgery and placement of the IOL. For example, a patient could end up much more myopic than desired. Again, an IOL exchange would be considered. Third, multifocal and accommodating IOL's have much less room for error when determining the proper lens for placement in the eye. Thus, in the case where a multifocal or accommodating IOL is placed, and the power is not exactly correct, the patient may be dissatisfied. It is challenging to remove and replace intraocular lenses generally, and the challenges are even greater with premium IOL's. A new approach to simply correct these issues with a minimally invasive procedure would add significant value to the surgical choices cataract surgeons can offer their patients. Another situation where such an approach would prove useful is in the setting of monocular cataract or asymmetric cataract in a subject with a refractive error. In order to avoid binocular diplopia or aniseikonia the power differences between the lenses of the two eyes should be roughly 2.5 diopters or less. Thus, a significant issue is exemplified by the following case. A bilateral −6.0 diopter myope has an asymmetric cataract. Cataract extraction is indicated for one eye only. Under this scenario, the cataract surgeon can at best leave the surgerized eye at a correction of −3.0 diopters so that binocular vision can be tolerated. However, the optimal long term result for this subject once the other cataract matures and is operated is to have each eye corrected close to plano. Because of this issue, sometimes cataract surgery is performed in both eyes, even the eye without a visually significant cataract so that a proper bilateral correction is placed. There would be value in allowing the cataract surgeon to operate only on the affected eye, correct it with an IOL close to the other eye's native power, and then when the second eye is operated (perhaps years later), a simple corrective procedure can be performed to correct the first eye's refractive error so the end result is satisfactory correction at distance for both eyes (plano, slightly myopic, or whatever the patient or surgeon prefers). Another situation where this invention may be of benefit is following scleral buckling surgery whereby the eye is elongated and the eye made more myopic for a pseudophake—the present approach can be a preferred alternative to additional spectacle myopic correction. Such an advance would give added flexibility and safe options to the cataract surgeon and patient with cataract.

The present invention describes a novel apparatus and lens for treating millions of individuals suffering from presbyopia following cataract or IOL surgery. The same approach (instrumentation and lens) provides a simple and minimally invasive surgical technique to correct situations where the IOL power needs changing, and in some embodiments also reduces the need for precise refractive correction.

One might ask, what is wrong with current piggyback lens technology? Can't a simple piggyback lens do the same thing? The answer is no. The key to success here is that the novel lens system has a protrusion that can coapt, fasten, or fit directly into the already secure IOL (the lens can be in the anterior chamber, posterior chamber, sulcus, capsular bag). An important component for the optics of presbyopic correction as well as corrective lens technology in general is optical system alignment. Furthermore, there is no currently available and appropriately sized instrument to allow for the insertion of the novel lens and simultaneous placement. Finally, the approach involves a method for creating a hole or penetration through the original IOL, and there is no available method or instrument that can make that hole for the joining of the two lenses. The approach described includes and instrument, a method with an energy source to create a place for the second novel lens to attach to the original IOL, and also a new novel intraocular lens with a protrusion specifically designed to attach to an IOL in an eye. Importantly, this approach allows for the proper centration of the second lens in the visual axis. Furthermore, the novel lens may in some embodiments utilize expandable materials such as expandable hydrogels in some or all of the aspects of the design so that once placed, the protrusion will swell, or expand, and create a secure attachment. The small, thin, and partially or dehydrated protrusion can be placed through a small hole with minimal excess force on the original IOL, making the insertion safe, and the expansion thus can atraumatically create a very strong attachment between the two lenses. Another important aspect to the technology described herein is the double barrel shape of the lens insertion instrumentation. By having one conduit for fluid, in some embodiments, that aspect of the device will allow for a stable anterior chamber and minimize the number of times an instrument must be placed into the eye. For example, in some embodiments a sequence of keratotomy, viscoelastic injection, and then energy source to create the site for fastening, and then lens inserter placement is not required. In some embodiments, one instrument can be inserted for the entire small incision procedure. In other embodiments, some of these steps may be required, but the lens and instrument are still novel. Note that the protrusion or fastening apparatus may be in the center of the novel lens, or off center, or toward the periphery of the lens. The protrusion may be round, cylindrical, square, rectangular, “T” shaped, oval, linear, or “L” shaped, or any variation thereof. There may be one or two or multiple protrusions emanating from the back surface of the secondary lens. The size and shape of the optic may vary as well. In some cases, such as for treating presbyopia the center lens may be small (1, 2 or 3 mm. or somewhere between 1 and 3 mm. in diameter for example), or larger for treating IOL power corrections. For IOL power corrections, the optic may be any diameter between 2 and 9 mm. The thickness may vary from anywhere between a fraction of a mm. to 1 to 2 mm. thick. In one embodiment the instrument and energy source can simply create a small (for example between 0.1 mm. and 2 mm. hole in the visual axis of the IOL creating a pinhole effect to allow for an adequate treatment for presbyopia or IOL power irregularities without a secondary lens.

It is thus an object of the present invention to provide an apparatus and method for treating presbyopia in a pseudophakic patient or treating improper IOL power problems in a pseudophakic patient. This approach is also amenable to utilization and can be performed in locations without extensive cataract surgical treatment support infrastructure. For example, a procedure room as compared to an operating room could be acceptable for the treatments described herein. It is another object of the present invention to provide an apparatus and method for treating presbyopia or conditions where the IOL power needs changing that can be performed in locations with fewer specialized practitioners. It is another object of the present invention to provide an apparatus to treat presbyopia in a pseudophakic patient. It is yet another object of the present invention to provide an apparatus to assist with a novel method of treating presbyopia. It is yet another object of the present invention to allow a surgeon to more easily and safely correct situations with incorrect IOL power in an eye.

There is also an opportunity for multiple aspects of this set of inventions to treat cataract (not pseudophakic presbyopia or IOL power problems, but lenticular opacity) along the lines discussed in previous patents and applications of a set of inventions claimed by the same inventor, and those filed documents are incorporated by reference herein.

### BRIEF SUMMARY OF THE INVENTION

In accordance with the present invention, there is provided an apparatus for treating presbyopia of a pseudophakic eye, cataract, or for situations where the IOL power needs changing, comprising a surgical tool formed with an angle to allow entry of the surgical tool through the peripheral cornea of the eye where the angle is formed by the convergence of a shaft for surgical handling and an operative head for surgical placement of a secondary intraocular lens; the operative head may have a sharp tool for penetration of a peripheral cornea of the eye; a delivery conduit disposed within said surgical tool for delivery of the secondary intraocular lens system device to a hole in a primary intraocular lens; and a guiding structure attached to the lens system device and further disposed within the surgical tool. The angle is defined by a line parallel with the operative end and the surgical end. There is no stipulation as to the radius of curvature. The guiding system may be removable, and the instrument can also allow for passage of a device or instrument that can create a hole in a cataract or IOL. The secondary intraocular lens may comprise a lens affixed to a retention structure; the retention structure having a generally cylindrical form and having a first end and a second end; the first end of the retention structure perpendicularly affixed to the lens; and a head having a diameter greater than the diameter of the retention structure and affixed to the second end of the retention structure. The surgical tool may be double barreled. The surgical tool may be triple barreled. One conduit may be used for infusion of saline solution. One conduit may be used for an energy source, one conduit may be used for the lens insertion, any combination thereof is considered and claimed as an invention here. The surgical tool may allow for an energy source, such as heat or diathermy or laser or other to create a hole

The foregoing paragraph has been provided by way of introduction, and is not intended to limit the scope of the invention and its various embodiments described, depicted, or envisioned herein.

### BRIEF DESCRIPTION OF THE DRAWINGS

The invention will be described by reference to the following drawings, in which like numerals refer to like elements, and in which:

FIG. 1 is a perspective view of a surgical tool of the present invention;

FIG. 2 is a rotated perspective view of the surgical tool of FIG. 1;

FIG. 3 is a plan view of the surgical tool of FIG. 1;

FIG. 4 is a rotated plan view of the surgical tool of FIG. 1;

FIG. 5 is a further rotated plan view of the surgical tool of FIG. 1;

FIG. 6 is a cross sectional view of the surgical tool of FIG. 1 cut along line B-B of FIG. 5;

FIG. 7 is an alternative embodiment of the surgical tool of the present invention;

FIG. 8 is a cross sectional view of the surgical tool of FIG. 7 cut along line C-C of FIG. 7.

FIG. 9 is another alternative embodiment of the surgical tool of the present invention;

FIG. 10 is a cross sectional view of the surgical tool of FIG. 9 cut along line D-D of FIG. 9;

FIG. 11 is a top plan view of the surgical tool of FIG. 1;

FIG. 12 is a bottom plan view of the surgical tool of FIG. 1;

FIG. 13 is a cross sectional view of the surgical tool of FIG. 1 cut along line A-A of FIG. 4;

FIG. 14 is a cross sectional view of the surgical tool of FIG. 1 cut along line B-B of FIG. 5 and having a lens system and guide installed;

FIG. 15 is a lens and fixture of the present invention;

FIG. 16 is a perspective view of the lens and fixture of FIG. 15;

FIG. 17 is a cross sectional view of the lens and fixture of FIG. 15 cut along line E-E of FIG. 15;

FIG. 18 is an exploded view of a lens of the present invention;

FIG. 19 is an exploded view of another embodiment of a lens of the present invention;

FIG. 20 is an exploded view of a lens, guide and retainer of the present invention;

FIG. 21 is a perspective view of the lens, guide and retainer of FIG. 20;

FIG. 22 is a cross sectional view of the lens, guide and retainer of FIG. 20 cut along line F-F of FIG. 20;

FIG. 23 is a plan view of a lens of the present invention;

FIG. 24 depicts an eye with an installation step of the lens of the present invention;

FIG. 25 depicts an eye with an installation step of the lens of the present invention;

FIG. 26 depicts an eye with an installation step of the lens of the present invention;

FIG. 27 depicts an eye with an installation step of the lens of the present invention;

FIG. 28 depicts an eye with an installation step of the lens of the present invention;

FIG. 29 depicts an eye with an installation step of the lens of the present invention;

FIG. 30 depicts an eye with an installation step of the lens of the present invention;

FIG. 31 is a plan view of a commonly used intraocular lens;

FIG. 32 is a perspective view of a commonly used intraocular lens;

FIG. 33 is a side view of a commonly used intraocular lens;

FIG. 34 is a rotated side view of a commonly used intraocular lens;

FIG. 35 is a plan view of a commonly used intraocular lens readied for placement of a secondary intraocular lens of the present invention;

FIG. 36 is a side plan view of the secondary intraocular lens of the present invention;

FIG. 37 is a perspective view of the secondary intraocular lens of the present invention;

FIG. 38 is a rotated side plan view of the secondary intraocular lens of the present invention;

FIG. 39 is a top plan view of the secondary intraocular lens of the present invention;

FIG. 40 is a side plan view of the secondary intraocular lens of the present invention cut along line G-G of FIG. 39;

FIG. 41 is a side plan view of the secondary intraocular lens of the present invention in a folded position;

FIG. 42 is a side plan view of the secondary intraocular lens of the present invention in a folded position cut along line H-H of FIG. 41;

FIG. 43 is a bottom plan view of the secondary intraocular lens of the present invention in a folded position;

FIG. 44 is a top plan view of the secondary intraocular lens of the present invention in a folded position;

FIG. 45 is a perspective view of the secondary intraocular lens of the present invention in a folded position;

FIG. 46 is a rotated perspective view of the secondary intraocular lens of the present invention in a folded position;

FIG. 47 is a plan view of a commonly used intraocular lens with the secondary intraocular lens installed therein;

FIG. 48 is a perspective view of a commonly used intraocular lens with the secondary intraocular lens installed therein;

FIG. 49 is a side plan view of a commonly used intraocular lens with the secondary intraocular lens installed therein;

FIG. 50 is a rotated side plan view of a commonly used intraocular lens with the secondary intraocular lens installed therein;

FIG. 51 depicts an eye with an installation step of the secondary intraocular lens of the present invention;

FIG. 52 depicts an eye with an installation step of the secondary intraocular lens of the present invention;

FIG. 53 depicts an eye with an installation step of the secondary intraocular lens of the present invention;

FIG. 54 depicts an eye with an installation step of the secondary intraocular lens of the present invention;

FIG. 55 depicts an embodiment of the present invention having an energy source;

FIG. 56 depicts various embodiments of the present invention; and

FIG. 57 depicts various embodiments of lenses of the present invention.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, claims and the attached drawings.

### DESCRIPTION OF THE PREFERRED EMBODIMENTS

For a general understanding of the present invention, reference is made to the drawings. In the drawings, like reference numerals have been used throughout to designate identical elements.

The present invention and the various embodiments described or suggested herein rely on a surgical technique where the natural lens of the eye or an intraocular lens installed within the eye is pierced and the resulting opening is mechanically retained and may contain a secondary lens. The resulting passageway lumen, or lens system created in the natural, cataractous or intraocular lens allows light to reach the retina, corrects for any existing lens discrepancies, thus improving vision. The resulting passageway through the natural lens or intraocular lens is made through a diameter less than the diameter of the natural lens. The use of this lens system device with or without the pinhole principle provides for a much simpler surgical technique and reduces related pre and post operative procedures.

The apparatus and method of the present invention may involve human cases, and also may be critically important in pediatric cases, as light will be in focus on the retina, reducing the need for complex cataract surgery followed by contact lenses or glasses in infants, and thus also may play a role in preventing amblyopia. In addition, veterinary cases of cataract such as those which develop in dogs, cats, horses, and the like, may benefit from the apparatus and method of the present invention and the various embodiments described or envisioned herein. The apparatus and method of the present invention may be critically important as an approach to pseudophakic presbyopia. The apparatus and method of the present invention may be critically important as an approach to situations where a premium IOL or single power standard IOL have created a refractive error that is unsatisfactory for a patient.

Turning now to the drawings, both an exemplary surgical device, as well as related devices and methods, will be described using the drawings attached herein. In these drawings, there is mention of two conduits, a delivery conduit and an infusion conduit. Those terms in some embodiments may be interchanged. In some embodiments there are three conduits. In some embodiments there is one conduit. In some cases a single conduit can serve multiple purposes, i.e. infusion and delivery of a lens or instrument. In the lens drawings there is a protrusion. The protrusion is depicted primarily as being round and in the center of the IOL. The protrusion in fact may be in any shape and may be central, off center, or peripherally located. The protrusion may be parallel to the optic, or at any angle ranging from parallel to perpendicular.

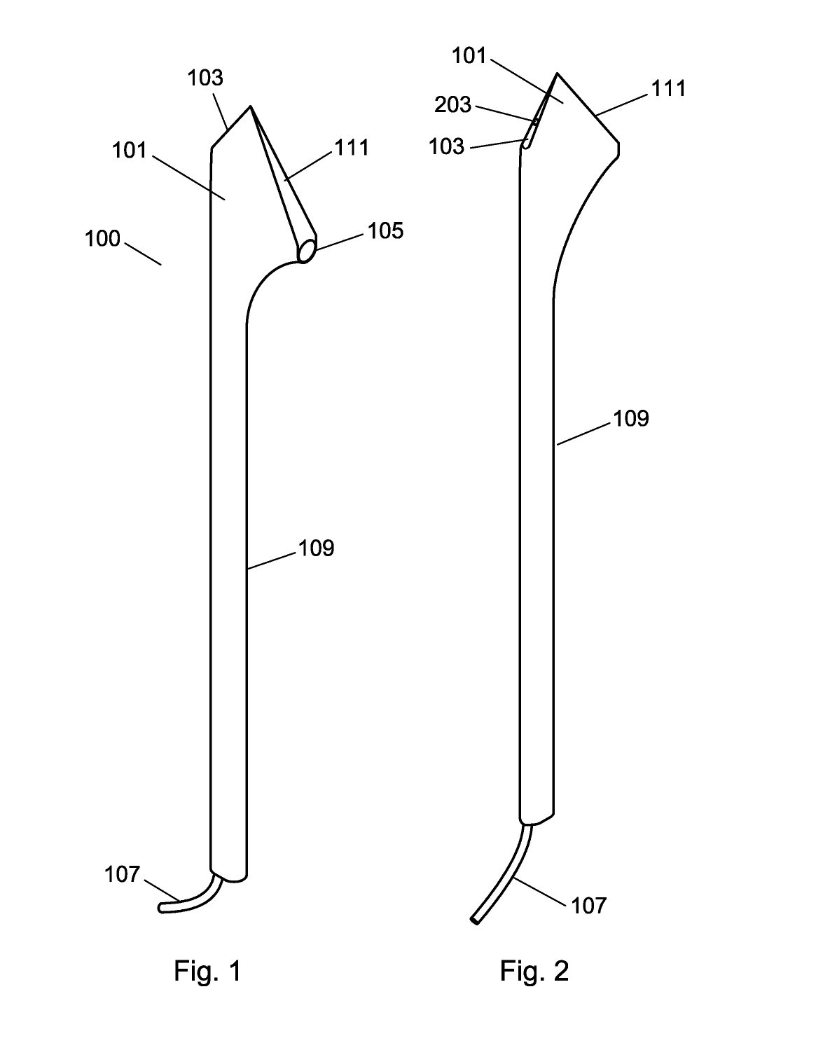

The surgical tool is depicted in the figures with an angle. The surgical tool, in some embodiments of the present invention, has no angle; it is generally straight or an angle as described may be less than 30 degrees, or in some embodiments of the present invention the angle is five degrees or less. In FIG. 1, a perspective view of a surgical tool of the present invention 100 is depicted. The surgical tool 100 may be made from a surgically compatible material such as, for example, stainless steel. The surgical tool 100 may be machined, cast, or the like. The surgical tool 100 is formed with an angle to allow entry of the surgical tool 100 through the peripheral cornea of the eye where the angle is formed by the convergence of a shaft 109 for surgical handling and an operative head 101 for surgical placement of a secondary intraocular lens. The surgical tool has a first edge 103 and a second edge 111. A delivery conduit 601 as seen in FIG. 6 has a delivery conduit exit 105 for deployment of a secondary intraocular lens in a primary intraocular lens of a pseudophakic eye. The shaft 109 may be rounded, oval, square, octagonal, or other such geometries. The operative head 101 may be joined with the shaft 109 at an angle such as ninety (90) degrees, or within sixty (60) degrees greater or less than ninety degrees, and may, in some embodiments of the present invention, have a radius at the angle. In some embodiments of the present invention, an irrigation conduit may travel the length of the shaft 109 and exit at the irrigation conduit exit 203 as seen in FIG. 2. An irrigation conduit fitting 107 is in fluid communication with the irrigation conduit (not shown in FIG. 1, FIG. 6, item 603). The drawings show two conduits. The device may have one or three conduits. The conduits may have specific unchangeable roles, or may be interchangeable. In one embodiment there is one infusion conduit and one conduit for an energy source on a guiding structure, and another conduit for a lens on a guiding structure. One or two of these conduits may be combined or even excluded. The operative head 101 has a sharp tool for penetration of a peripheral cornea of the eye in the diagram. The instrument may or may not have a sharp tool for cutting. The sharp tool may have a different or smaller appearance. The sharp tool may be placed near, or a part of, the first edge 103 or the second edge 111.

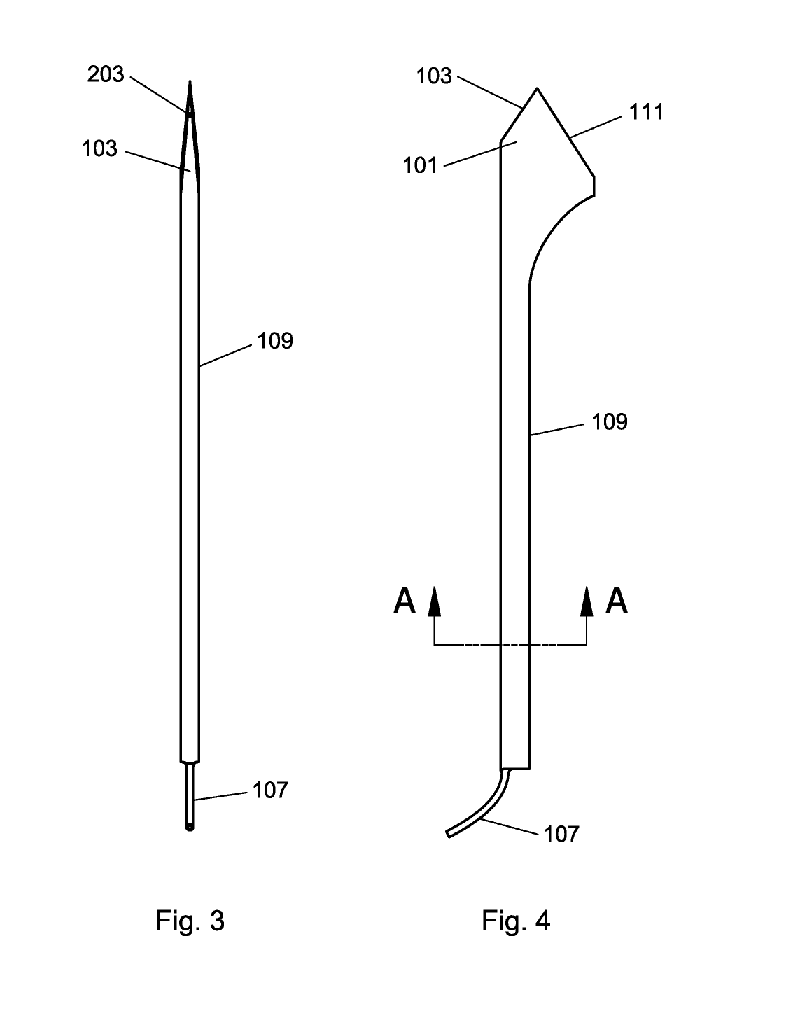

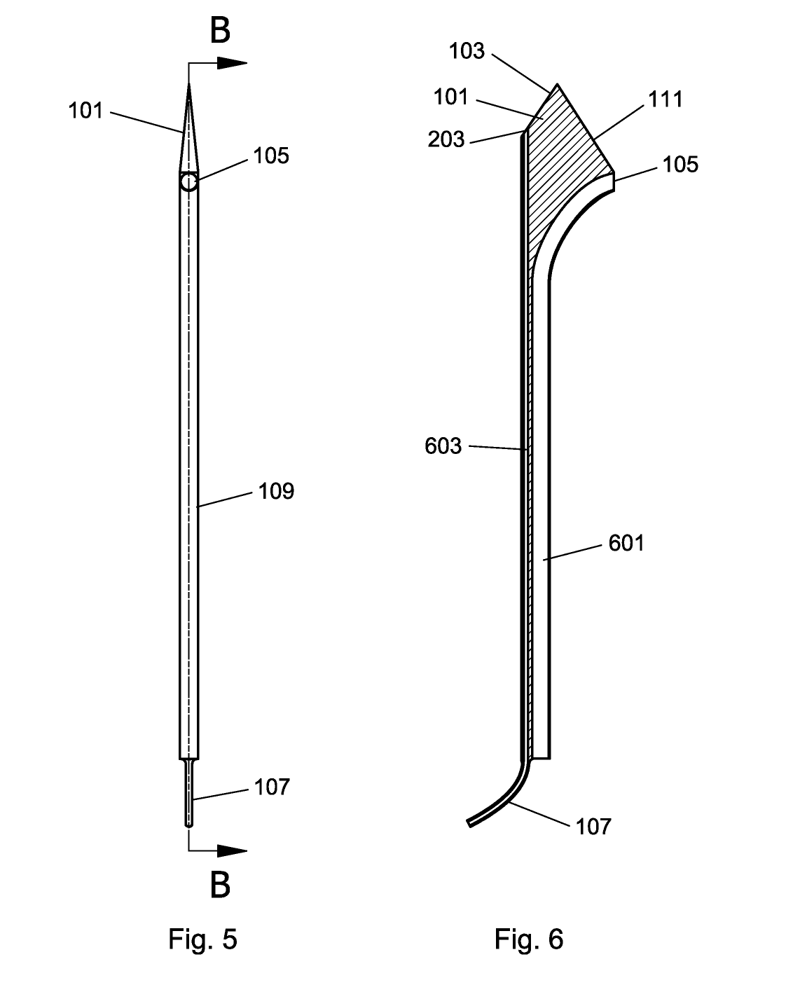

FIG. 2 is a rotated perspective view of the surgical tool of FIG. 1 where the irrigation conduit exit 203 can be seen at the first edge 103. FIG. 3 is a plan view of the surgical tool of FIG. 1 where the first edge 103 can be seen. The first edge 103 comes to a point at the distal end of the operative head 101. FIG. 4 is a rotated plan view of the surgical tool of FIG. 1. FIG. 5 is a further rotated plan view of the surgical tool of FIG. 1 where the delivery conduit exit 105 can be seen. FIG. 6 is a cross sectional view of the surgical tool of FIG. 1 cut along line B-B of FIG. 5. The delivery conduit 601 can be seen as it travels along the shaft 109 to its point of exit 105. The irrigation conduit 603 can also be seen as it travels along the shaft 109. The irrigation conduit fitting 107 may be connected to an external source of fluid by way of a hose, tube, conduit, or the like.

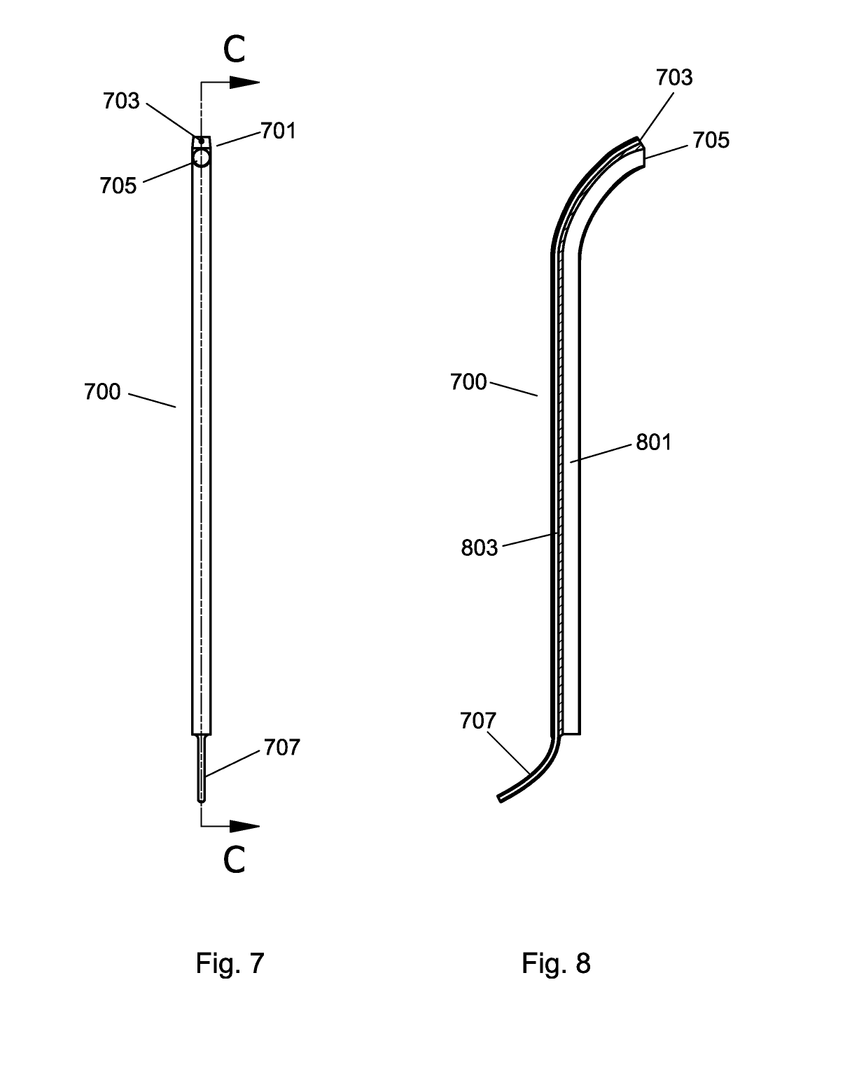

FIG. 7 depicts an alternative embodiment of the surgical tool of the present invention with a modified operative head 701. The surgical tool 700 is formed with an angle to allow entry of the surgical tool 700 through the peripheral cornea of the eye where the angle is formed by the convergence of a shaft for surgical handling and an operative head 701 for surgical placement of a secondary intraocular lens. The operative head 701 has a sharp tool for penetration of a peripheral cornea of the eye. The sharp tool has a multi-angled surface, such as a two angle surface. A delivery conduit 801 (see FIG. 8) is disposed within said surgical tool 700 for delivery of the secondary intraocular lens system device to a hole in a primary intraocular lens. The conduit may be used to pass an energy source on a guiding structure as well. A separate similar conduit may exist alongside the conduit pictured creating a third conduit in the surgical tool. There may be one, two or three conduits. The conduits may allow for the delivery of an energy source on a guiding structure and/or a lens on a guiding structure. Irrigation may be delivered through one or all conduits. The conduits may open into the same area of the surgical tool, or as depicted on a different or opposing side. In the operative head 701 an irrigation conduit exit 703 and a delivery conduit exit 705 can be seen along with an irrigation conduit fitting 707 for connection to an external source of fluid by way of a hose, tube, conduit, or the like. FIG. 8 is a cross sectional view of the surgical tool of FIG. 7 cut along line C-C of FIG. 7. The delivery conduit 801 can be seen as it travels along the shaft to its point of exit 705. The irrigation conduit 803 can also be seen as it travels along the shaft. The irrigation conduit 803 may be absent from some embodiments of the present invention, or may be modified or altered from that depicted in the drawings.

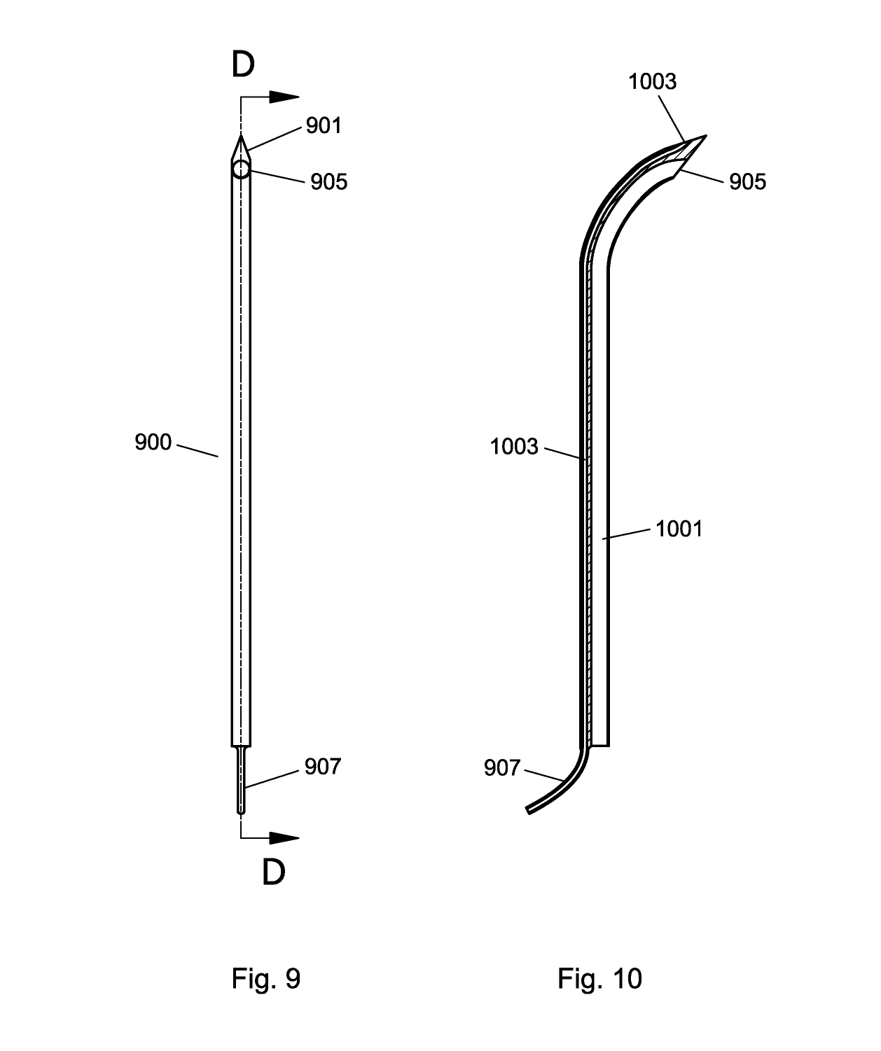

FIG. 9 depicts another alternative embodiment of the surgical tool of the present invention 900. The surgical tool 900 is formed with an angle to allow entry of the surgical tool 900 through the peripheral cornea of the eye where the angle is formed by the convergence of a shaft for surgical handling and an operative head 901 for surgical placement of a secondary intraocular lens. The operative head 901 has a sharp tool for penetration of a peripheral cornea of the eye. The sharp tool has a single angled surface. A delivery conduit 1001 (see FIG. 10) is disposed within said surgical tool 900 for delivery of the secondary intraocular lens system device to a hole in a primary intraocular lens. In the operative head 901 a delivery conduit exit 905 and an irrigation conduit 1003 can be seen along with an irrigation conduit fitting 907 for connection to an external source of fluid by way of a hose, tube, conduit, or the like. There again may be three conduits, one, or two conduits. The conduits may allow for the delivery of an energy source on a guiding structure and/or a lens on a guiding structure. Irrigation may be delivered through one or all conduits. The conduits may open into the same area of the surgical tool, or as depicted on a different or opposing side. FIG. 10 is a cross sectional view of the surgical tool of FIG. 9 cut along line D-D of FIG. 9. The delivery conduit 1001 can be seen as it travels along the shaft to its point of exit 905. The irrigation conduit 1003 can also be seen as it travels along the shaft. The irrigation conduit 1003 may be absent from some embodiments of the present invention, or may be modified or altered from that depicted in the drawings.

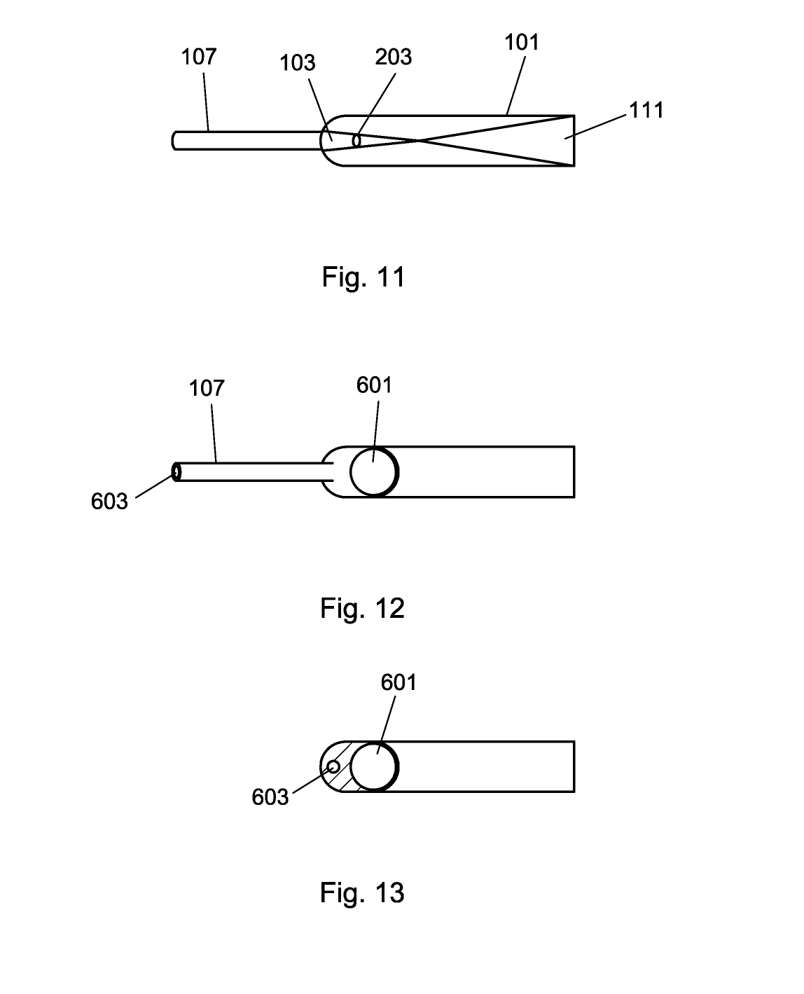

Turning now to FIG. 11, a top plan view of the surgical tool of FIG. 1 is shown. The first edge 103 and the second edge 111 can be seen along with the irrigation conduit exit 203 and the overall operative head 101. FIG. 12 is a bottom plan view of the surgical tool of FIG. 1 where both the delivery conduit 601 and the irrigation conduit fitting 107 can be seen. FIG. 13 is a cross sectional view of the surgical tool of FIG. 1 cut along line A-A of FIG. 4. Both the delivery conduit 601 and the irrigation conduit 603 can be clearly seen. There again may be three conduits, one, or two conduits. The conduits may allow for the delivery of an energy source on a guiding structure and/or a lens on a guiding structure. Irrigation may be delivered through one or all conduits. The conduits may open into the same area of the surgical tool, or as depicted on a different or opposing side.

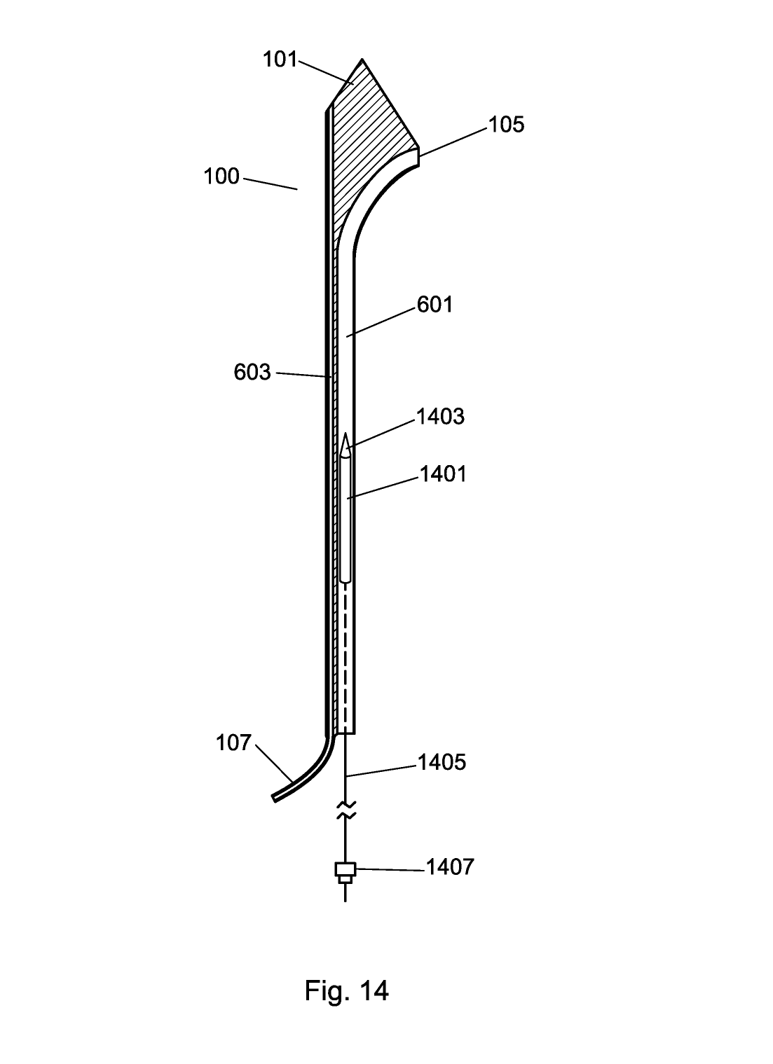

FIG. 14 is a cross sectional view of the surgical tool of FIG. 1 cut along line B-B of FIG. 5 and having a lens system and guide installed. The conduit may allow for the delivery of an energy source on a guiding structure as well as a lens on a guiding structure. Irrigation may be delivered through one or all conduits. The conduits may open into the same area of the surgical tool, or as depicted on a different or opposing side. The lens 1401 can be seen in the delivery conduit 601. The lens depicted may be a tubular lens to pierce an IOL or cataract, or it may have a protrusion that allows for the attachment of the lens to a primary IOL already in place inside the eye. In some embodiments of the present invention, the lens 1401 has a taper 1403 to allow for easier guidance and entry of the lens 1401. The lens 1401 has attached thereto a guide wire 1405 that may also have, in some embodiments of the present invention, a guide tool 1407 at one end of the guide wire to facilitate manipulation of the lens 1401 by way of mechanical coupling through the guide wire 1405. Prior to the use of the lens and guiding instrumentation, the conduit or a separate conduit may be used to create a hole in the primary IOL or cataract.

The guiding structure, or guide wire in this example 1405 must be capable of traveling through the angle in the delivery conduit 601 at the juncture of the operative head 101, and must convey force through this angle, and must be of sufficient rigidity to support the insertion of the lens 1401 into an intraocular lens. Materials for the guide wire may include stainless steel, titanium, Kevlar, encapsulated glass (fiber optic material), and the like. Materials for the guiding structure may include metals, plastics, polymers, and the like. The guiding structure term is nomenclature that may include, but is not limited to, a guiding or lens system advancing structure, and also serves to reference a system that would deploy through the delivery conduit 601 for the purpose of piercing a lens such as an intraocular lens allowing for the placement of the lens 1401. The guiding structure may also refer to a method to create a hole or penetration allowing or helping to advance the lens system device into the intraocular lens. Heat, electrocautery, diathermy, ultrasound, laser, or other electromechanical processes may be deployed at the tip to ease the penetration of the system through the lens. The material or part of it, such as, for example, the leading or trailing ends, may also be biodegradable material such as Polyglycolic acid polymers. The lens may be foldable, bendable, and expandable. It may be molded of a single material or a molded dual or three material lens.

In addition, the lens 1401 may further contain drugs that are carried in a polymeric coating or sleeve that may or may not be loaded with at least one therapeutic drug. Suitable polymers for the lens system device, with or without drug loading include, for example, poly(methyl methacrylate) (“PMM.A”), poly(ethylene-co-vinyl alcohol) (“EVAL”), poly(butyl methacrylate) (“PBMA”), polyglycolic acid (“PGA”), poly(L-lactic acid) (“PLLA”), silicones, hydrogels, copolymers and blends thereof, as well as various nanomaterials such as carbon nanotubes and the like. The lens or lens system device may also be made from a hydrophilic material that expands upon placement in an aqueous environment such as an eye. Suitable hydrophilic materials may include, but are not limited to, Hema (Poly-hydroxy-ethylene thacrylate), 2-hydroxyethyl methacrylate (HEMA), hydrophilic acrylic, 2 HEMA hydrophilic acrylic, hydrogels, hydrophilic polymers, fluorocarbon-sulfone. Thus, a dehydrated transparent hydrophilic polymer would expand over time once placed in position through the cataract. Any transparent and biocompatible material from which a contact lens or intraocular lens can be made is embodied in the lens designs and materials herein. Hydrophobic acrylic or silicone lenses may also be used. The secondary lens may be single power, toric, or custom, meaning the lens has corrections for higher order aberrations. The lens may correct for astigmatism.

Also depicted in FIG. 14 and later figures of the lens system device is a taper 1403 that is an integral part of, or a detachable part of, the lens system device 1401. The taper 1403 allows the lens system device 1401 to travel and insert into the created lumen in an in situ cataract, or in some cases, the intraocular lens. In some cases the taper may have a thinner space just proximal so that when the taper, or protrusion is placed through a primary IOL it will secure itself. This aspect of the device may also be capable of transmitting heat, ultrasound, laser, or other electromechanical energies to ease placement through the existing lens. This leading edge may be connected by a thin wire or guiding system that can be retracted while leaving the tubular IOL or secondary novel lens in place. The taper 1403 may be present in some embodiments of the present invention, and may be absent from the lens system device 1401 in other embodiments of the present invention. The taper may, in some embodiments of the present invention, be biodegradable, or threaded. In some embodiments of the present invention, the lens system device 1401 may be surrounded by a component that would prevent lens material from entering the patent space opened by the stent or energy source once it is removed. The component that surrounds the lens system device to keep the lens material out could be made from Dacron®, Gore-Tex®. Nylon, or other biodegradable or non-biodegradable materials. The component that inserts in the cataract, that keep lens material from disseminating into the eye could also be an independent structure, forming a hollow cylinder. It could be coated with the same biocompatible material. This outer coating, or the entire lens system device could be made from intraocular lens material such as acrylic, PMM.A, or silicon. The hollow cylinder may have structure supplied by materials such as metal, plastic, or biocompatible polymers. Nitinol is one metal of key applicability. The taper is of particular use when piercing an in situ cataract. It may also be helpful in placing a secondary IOL.

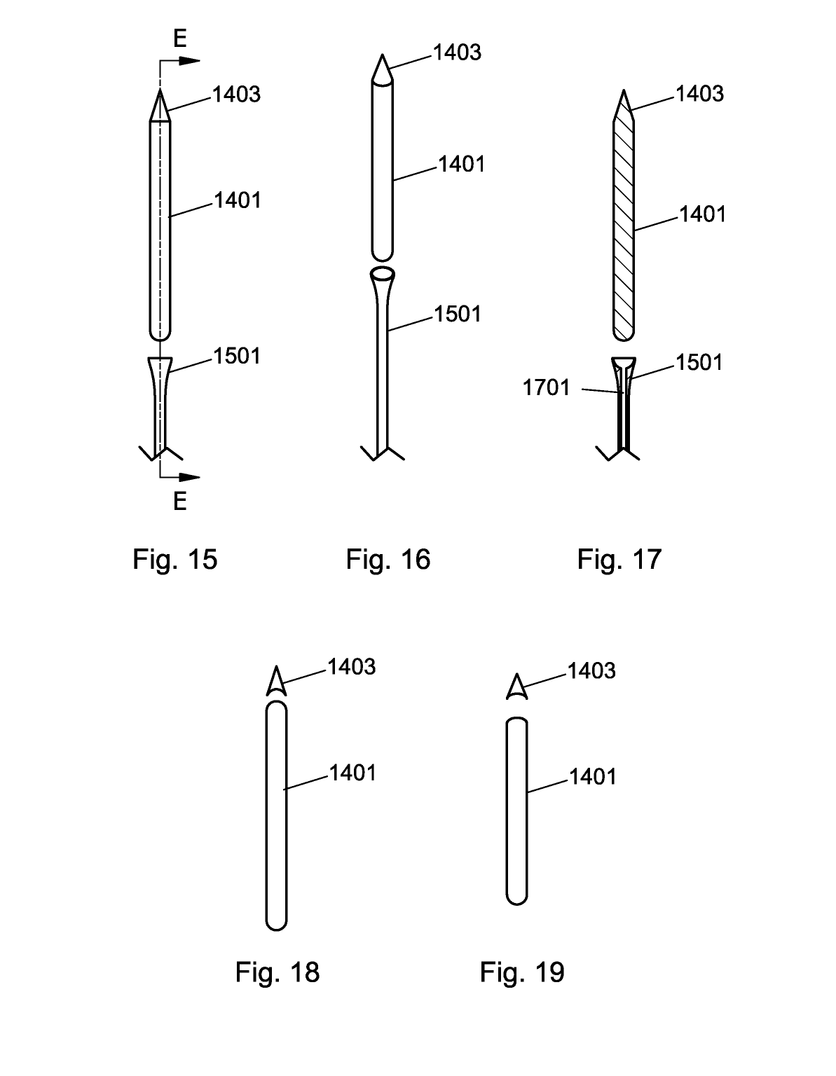

FIG. 15 is a lens and fixture of the present invention, the lens 1401 and taper 1403 having been heretofore described. A fixture 1501 such as a retention fixture can be seen in FIGS. 15-17. The fixture 1501 serves to releasably affix the lens 1401 to the guide wire (see FIG. 14) while the lens is being deployed. The fixture 1501 also serves to release the lens 1401 once in place in the intraocular lens. FIG. 16 is a perspective view of the lens and fixture of FIG. 15 also showing the fixture 1501 uncoupled from the lens 1401. FIG. 17 is a cross sectional view of the lens and fixture of FIG. 15 cut along line E-E of FIG. 15. In FIG. 17 a release via 1701 can be seen passing through the fixture 1501. The release via 1701 may provide suction or similar vacuum to the lens 1401 to provide releasable retention of the lens 1401. Upon proper placement of the lens 1401, the suction is stopped, and the lens 1401 is therefore released from the fixture 1501. In other embodiments of the present invention, the lens 1401 is retained to the fixture with an adhesive, friction, or the like.

FIG. 18 is an exploded view of a lens of the present invention and FIG. 19 is an exploded view of another embodiment of a lens of the present invention. The lens 1401 and the taper 1403 can be seen without a retainer. The drawing can also schematically represent, in addition to the tubular IOL, the secondary IOL with its protrusion leading the way distally.

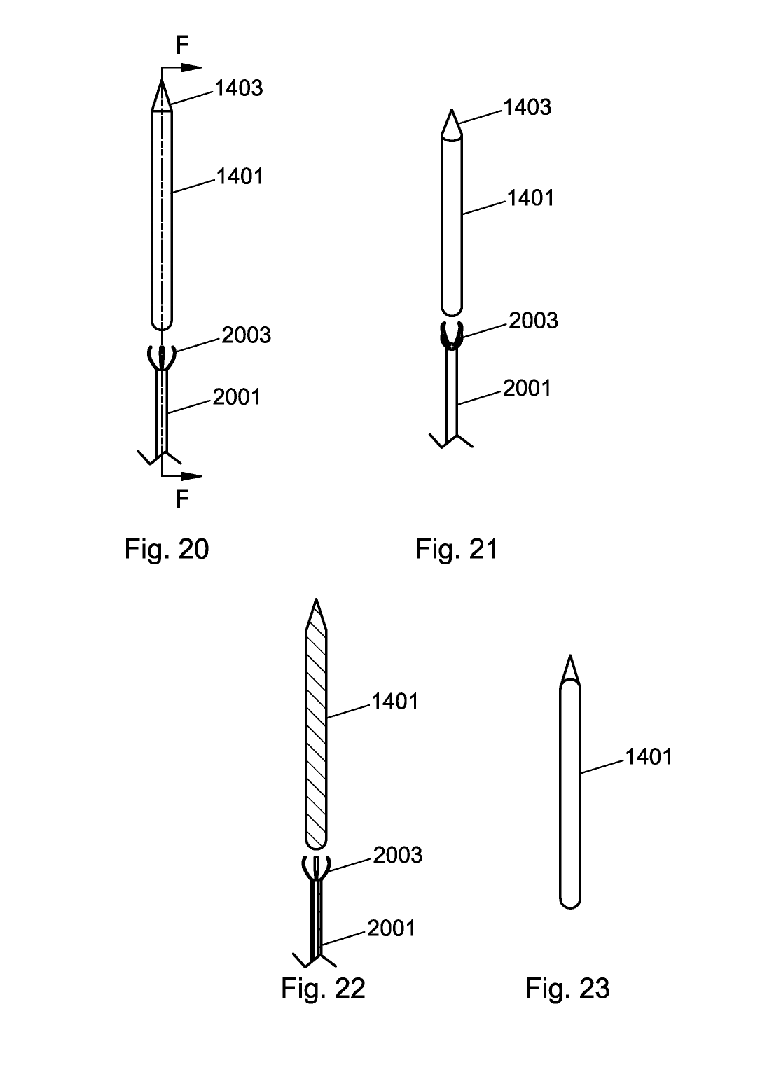

Various retainers may be employed with the lens system (tubular IOL or secondary IOL with a protrusion) of the present invention. These drawings can also schematically represent, in addition to the tubular IOL, the secondary IOL with its protrusion leading the way distally. For example, FIG. 20 is an exploded view of a lens, guide and retainer of the present invention. The retention structure 2003 of FIG. 20 comprises moveable prongs that grip the lens 1401 and are mechanically coupled to a guide 2001 such as, for example, a guide wire. The moveable prongs will either grip or release the lens 1401 upon the application of force to the guide in the appropriate direction. FIG. 21 is a perspective view of the lens, guide and retainer of FIG. 20 showing four prongs with the retention structure. In some embodiments of the present invention, more or less than four prongs may be employed. FIG. 22 is a cross sectional view of the lens, guide and retainer of FIG. 20 cut along line F-F of FIG. 20 showing the guide 2001 in mechanical communication with the prongs of the retention structure 2003. FIG. 23 is a plan view of a lens of the present invention without the guide or retention structure present.

The lens system device, surgical tool, and related methods, techniques and devices may be used to provide a restorative lens or passageway to a cataractous natural lens. An apparatus to treat cataract is described in U.S. Pat. No. 8,343,214 to Dr. David Kleinman and entitled “Apparatus For The Treatment of Cataract”, the entire disclosure of which is incorporated herein by reference in its entirety. The system that is described herein can also apply to placing a secondary IOL securely in position by fastening it to a primary IOL. The primary IOL could be single power, anterior chamber or posterior chamber, or a premium IOL.

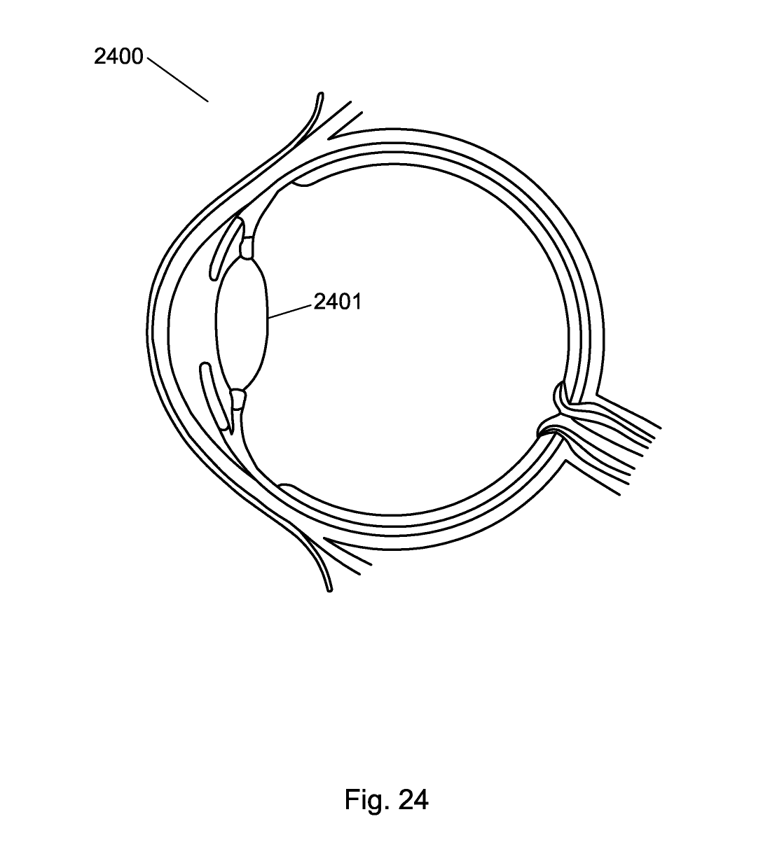

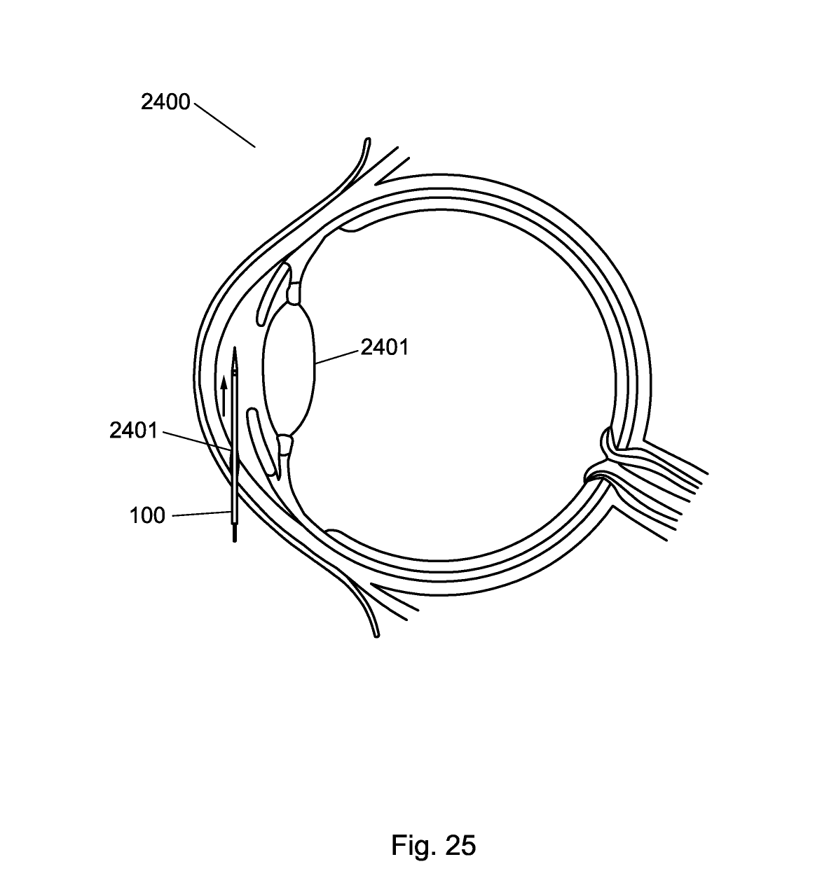

FIGS. 24-30 depicts an eye with an installation step of the lens of the present invention whereas the lens is installed in a cataractous natural lens.

FIGS. 24 through 30 will provide examples of the steps involved in the exemplary methods of the present invention, and also provide examples of how to use the surgical tool and related implantable device of the present invention. The examples provided in the figures are not to be construed as limitations of the present invention and its various embodiments described herein, but rather, are depicted to fully allow one skilled in the art to make and use the invention along with modifications, adaptations, and alterations, all being within the spirit and broad scope of the present invention as fully defined herein. FIGS. 24 through 30 depict a human eye (adult or pediatric), but the methods and apparatus of the present invention are equally well adapted to veterinary medicine. The invention may have particular applicability toward the treatment of pediatric cataract or congenital cataract. If there is an opacity (cataract) in the visual axis of an infant or child less than seven years of age, amblyopia in the affected may develop. The amblyopia could be severe and prevent the affected eye from ever seeing better than 20/200 or better than legal blindness in the United States. Current methods of treating pediatric cataract include removal of the lens (by phacoemulsification typically). Such an approach in an infant is often coupled with surgical aphakia. Contact lens care is required of the caregiver. This can be tedious and challenging in a young child, and despite best efforts, amblyopia can develop. Placing an IOL in an infant is possible, but it has its own issues including lens power choices, risk of complications, and refractive error. The current set of inventions and novel lenses and instruments can help reduce rates of amblyopia associated with pediatric cataract. For example, the tubular IOL could be placed through a pediatric cataract providing 20/20 correction at one meter or 0.5 meter allowing the child's visual system to develop, and then after several years, when the eye is nearly full grown, a procedure could be performed to remove the tubular IOL and place a standard IOL. In another, this time veterinary, example, dogs are prone to cataracts as they age, and canine cataract surgery tends to be costly, and many pet owners chose to not treat the cataracts of their beloved pet due to cost constraints. A lower cost, simpler solution to cataracts in pets is much needed.

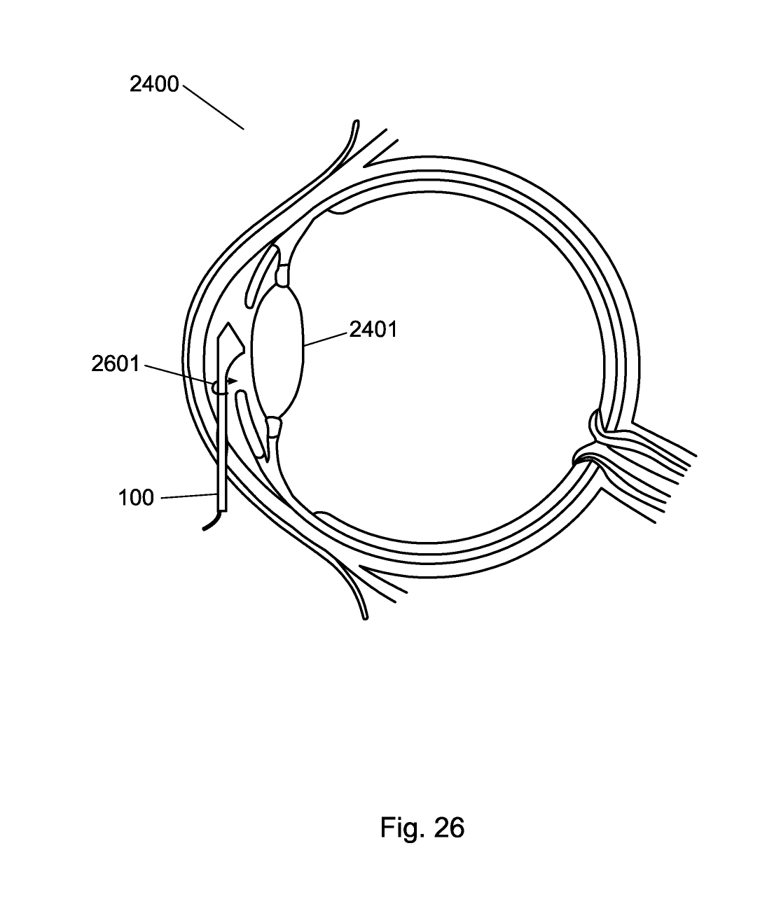

In FIG. 24, an eye 2400 is depicted showing a natural lens 2401. In FIG. 25, an incision 2501 is made in the cornea, the diameter of the incision through the cornea can be between 0.25 mm. and 2 mm. or 3 mm., being self sealing, but may be up to 6 mm., and may require surgical closure. In some embodiments of the present invention, a support structure or support structures may be used to hold the lens in place during surgery. In some embodiments of the present invention, a polymer block is used that rests externally on the eye to stabilize it so that when the guide (with or without an accompanying flexible or rigid catheter) and other related technologies are entered into the cornea, the eye is held steady to minimize the risk of injury through movement. Viscoelastic, or other viscous materials may be injected into the anterior chamber (and left in place or washed out) during the procedure to maintain the anterior chamber. Alternatively, a simple anterior chamber maintainer such as a needle or Lewicky cannula may be used to maintain the anterior chamber. The goal of the surgery is to place a hole and support structure through the cataractous lens 2401, thus allowing light to enter the back of the eye to the retina, improving vision. FIG. 25 now depicts the surgical tool 100 entering the incision 2501 in a linear direction of travel. Note that the surgical tool 100 is placed in such a way as to clear any and all obstructions. In FIG. 26, once the surgical tool 100 is in a position where the guiding structure or guide wire (not shown) may be directed toward the visual axis and center of the lens, the surgical tool 100 is rotated in a rotational direction of travel 2601 such that a pinhole and related lens system device deployment may be made. FIG. 26 shows the surgical tool in the proper position after rotation.

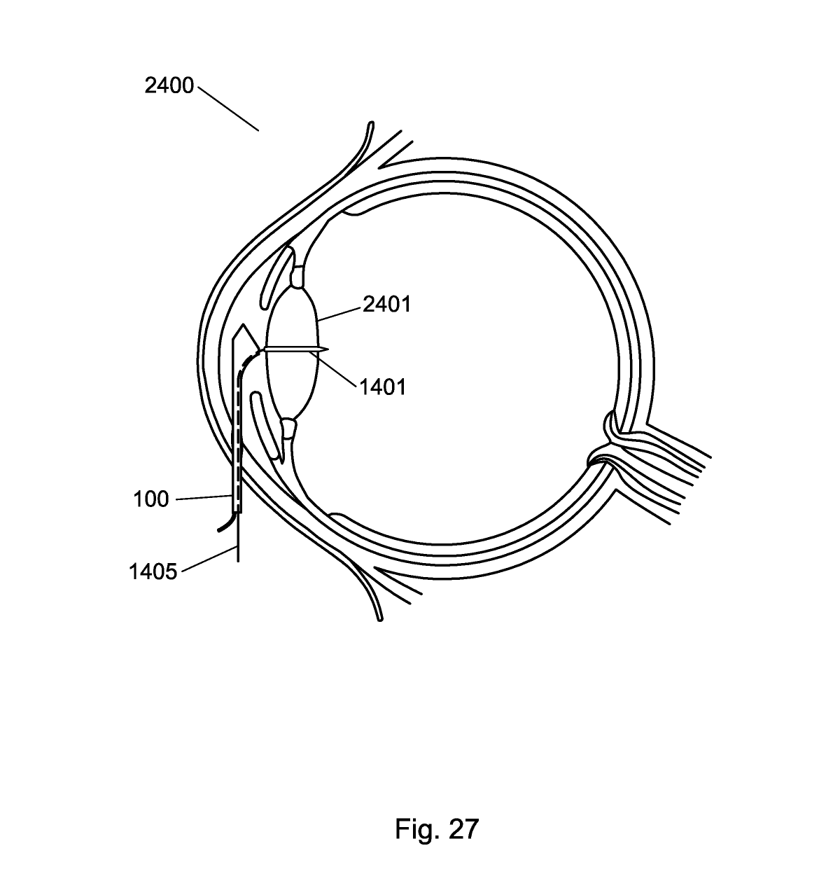

In FIG. 27, a guide wire or guiding apparatus 1405 or similar rigid structure is inserted in the delivery conduit or other hollow of the surgical tool 100, passed through the hollow of the surgical tool 100, and allowed to exit the surgical tool 100 in such as a way as to create a pinhole, track, passageway or lumen through the lens. The guide wire or guiding structure 1405 may include flexible wires, catheters, needles, hollow pipes, fiberoptic cables, rotational drills, ultrasound, electrocautery, lasers including femtosecond, continuous wavelength, ND YAG, and other methods to generate heat or other types of energy to pierce the anterior lens capsule and then the entire lens to create an opening through that lens (note that similar approaches may be used to create a hole in a primary IOL). Any or all of these techniques may also be used to create a space for insertion of a guide wire and with or without similar methods for the passage of the ultimate cannula, stent, lens system (expandable lens in some embodiments) that eventually maintains the patent opening through the cataract. In some embodiments of the present invention, a fiberoptic cable or strand may be used to maintain patency of the opening, and, in some embodiments of the present invention, may be used to bypass a cataract. In some embodiments of the invention the lens device system or stent is passed over the guiding structure into the lens for its deployment. In other embodiments of the present invention, the guiding structure and or guide wire may be passed into the lens simultaneously with the lens device system or stent, such that the guiding apparatus essentially allows for the positioning, deployment, and eventual release of the lens device system, expandable intraocular lens, stent, or lumen creation device. Once the hole, passageway lumen or tunnel is created in the lens, a stent assembly, or lens system 1401 or similar structure is conveyed to or thorough the pinhole using guiding techniques such as guiding structures and the like. Or, the guiding structure could simply advance the lens or stent system into the lens so that it can be deployed. The guiding system for the energy source may run parallel in a third lumen (conduit) or the same lumen. The lens system device and the guide for the energy source may be inserted in parallel and the energy source and guidewire may be removed leaving the lens in place. This guiding structure 1405 may also consist of a double barrel design or triple barrel design (one conduit inside another or side by side or one or the other or a combination thereof) whereby the inner guiding structure, or one conduit, is used to advance the lens piercing and/or lens device system into the cataract, and the outer portion is able to place counter traction on the lens device system, stent, or lumen such that the guiding structure can be removed, pulled away, and/or separated from the lens device system, stent, or lumen, and the in situ cataract without dislocating or otherwise destabilizing the new integrated relationship between cataract and lens device system, stent or lumen. Other release systems have been heretofore described and envisioned herein. Some release systems would allow for the separation of guiding structure and lens device structure via severing the guiding structure from the stent or lens device system anteriorly or proximal to the lens device system or stent. Methods to accomplish this separation include but are not limited to a triggered release system activated mechanically through, for example, suction, a fine wire, or though a micromachine or microelectromechanical system that can be turned on by a remote electromagnetic signal, or by a heat or electrically activated release mechanism whereby the portion of the guiding structure just proximal to the lens device system or stent is designed to melt, break, or become extremely weak to axial motion following activation. Thus, the lens device, system, lumen, expandable tubular lens, or stent could be left in place in the cataract after deployment or placement with minimal additional tissue trauma thereby reducing risks of lens subluxation, dislocation, or damage. The diameter of the initial piercing of the lens creating the pinhole, lumen, or tunnel, at first is typically between one and two millimeters, but may be between 0.10 mm. and 5 mm. In some embodiments of the present invention, the cornea may also be entered from the peripheral cornea using a guide to replace the surgical tool 100 that has memory to redirect the wire, catheter, and lens system device posteriorly through the lens so that the eye need not be entered through the central cornea. In such a case, there would be a small corneal incision made with a separate blade or needle, and then the lens device system/guiding structure with curvature memory could be advanced into the eye, or the straight guiding structure/lens device deployment system apparatus with memory for curvature may penetrate the outer cornea directly with a sharp tip. There may be a secondary aspect to such a straight placement system such that an outer shell or double barrel system allows for the memory or curvature to become manifest following the retraction or removal of the outer shell or outer hollow barrel. The stent or lens system device 100 is placed in or near the visual axis of the eye. In some embodiments of the present invention, the stent or lens system device 100 is placed outside the visual axis of the eye, but is still able to improve sight. In some embodiments of the present invention, the lens system device, stent, small diameter cannula or other pinhole device may be mechanically forced through the cataractous lens using a mechanical system such as a spring loaded mechanism or screw/threaded rotational mechanism or the like. A mechanism to force insertion of the lens system device, stent or other pinhole device may also be electrically or battery powered, and may, in some embodiments of the present invention, include a hand powered, mechanical or electrical system to drive the lens system device, stent or other pinhole device carefully through the lens. The mechanical energy system aspect of the device may utilize heat energy, ultrasound, laser, cautery, ionization, or other electromechanical energies to ease placement through the cataractous lens. There may be a drill, a laser, or a plasma knife involved. Such mechanisms may also be used, in some embodiments of the present invention, to drive the guide wire through the lens. In this described procedure., the pupil may be dilated or may be undilated. The procedure may be used with viscoelastic materials or without, and may include, in some embodiments of the present invention, a self healing incision. An irrigation system may be a part of the device or the deployment system of the present invention in some embodiments of the present invention. Some embodiments of the present invention may include an intraocular lens within the assembly. The intraocular lens is placed through the cataract, without removing the cataract. The intraocular lens will have a small diameter optic, for example, less than 3 mm. The lens may be flexible, expandable, or rigid, and may be preloaded in the surgical end of the operative tool or the operative end of the tool and then subsequently advanced through the bend in the tool during use. The same concept applies to any stent or lens system device that may be deployed by the apparatus—it may be preloaded in the surgical or operative end of the tool, or it may be secondarily advanced over a guide wire. Furthermore, after creating the opening through the cataract, a piggy back intraocular lens, or larger optic may be placed in the anterior chamber or in the anterior segment in front of the cataract but behind the iris to help focus light on the retina. The human lens, for reference, is 9 mm. in diameter and 4 mm. thick. Standard optics used in cataract surgery have an optic diameter between 6 mm. and 4 mm. In some embodiments of the present invention, the stent may be conical with the anterior opening larger than the posterior opening. A wide angle lens to allow for a larger field of view on the retina may be utilized in this system. The stent or intraocular lens may, in some embodiments of the present invention, have a flexible flange on the lead end and trailing end to help secure it in position. The intraocular lens may further, in some embodiments of the present invention, be telescoping. The lens system device may come in various powers to optimally correct for refractive error.

In FIG. 27, the lens 1401 is properly positioned in and through the cataract, and the guiding structure 1405 is retracted through the surgical tool 100. These methods may be performed by a physician a non-physician, a veterinarian, or the like, under topical, local, or general anesthesia. The methods may be performed with or without the use of viscoelastic material or balanced saline irrigation. The lens 1401 or stent or cable or cannula or catheter or clear tunnel or lens system or polymer lens extends through the entire lens of the eye. The length of this stent, cable, new lumen, lens system, or material can extend 3 mm. in front of the anterior surface of the lens and up to 18 mm. posterior to the posterior capsule of the lens. Typically the length will extend 0.5 to 2 mm. anteriorly and 0.5 to 3 mm. posteriorly through the lens. The lens 1401 may be an expandable stent, or a hydrophilic material that expands upon placement in an aqueous environment such as an eye. Suitable hydrophilic materials may include, but are not limited to, Hema (Poly-hydroxy-ethylene thacrylate), 2-hydroxyethyl methacrylate (HEMA), hydrophilic acrylic, 2 HEMA hydrophilic acrylic, hydrogels, hydrophilic polymers, fluorocarbon-sulfone. Any transparent and biocompatible material from which a contact lens or intraocular lens can be made is embodied in the lens designs and materials herein. Hydrophobic acrylic or silicone lenses may also be used.

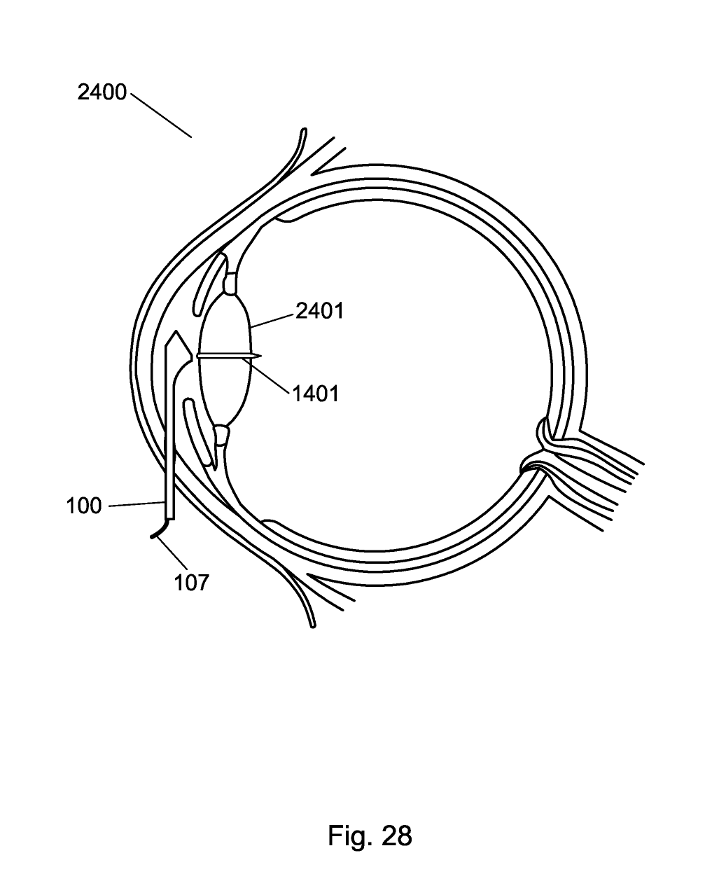

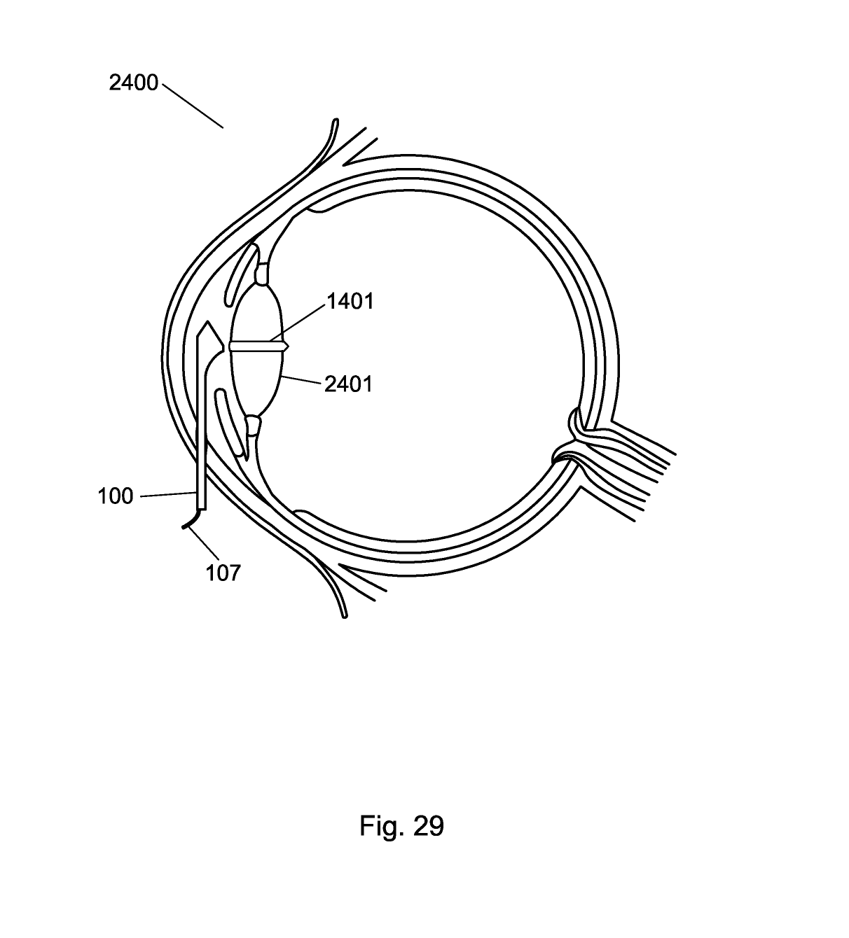

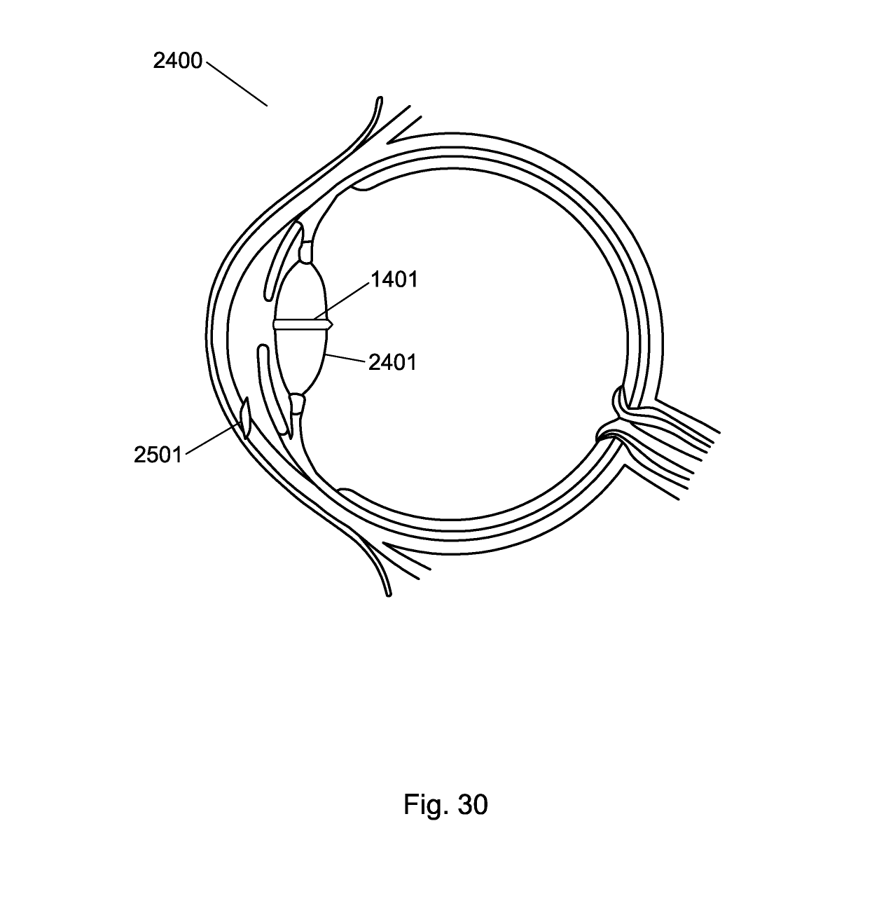

FIG. 28 shows the lens 1401 fully installed, with the guide wire 1405 removed. In FIG. 28, the lens 1401 has a taper 1403. Other embodiments of the present invention may have a modified taper, or no taper. The taper may have threads to assist its deployment into the cataractous lens, and the tapered aspects may convey other mechanical advantages. FIG. 29 depicts the lens 1401 fully installed and expanded. The taper may be detachable and biodegradable. The taper may contain an active pharmaceutical agent formulated in the taper for release inside the eye. Lastly, FIG. 30 depicts the lens 1401 fully installed and with the surgical tool 100 removed.

Light ray tracing analysis shows that a reasonable image can be formed on the retina using an intraocular pinhole system, and one embodiment of this invention is a pinhole intraocular lens system. An optical engineer using ray tracing technology can demonstrate that visual acuity potential is better than 20/100 and can be as good as 20/20 in tubular lens designs.

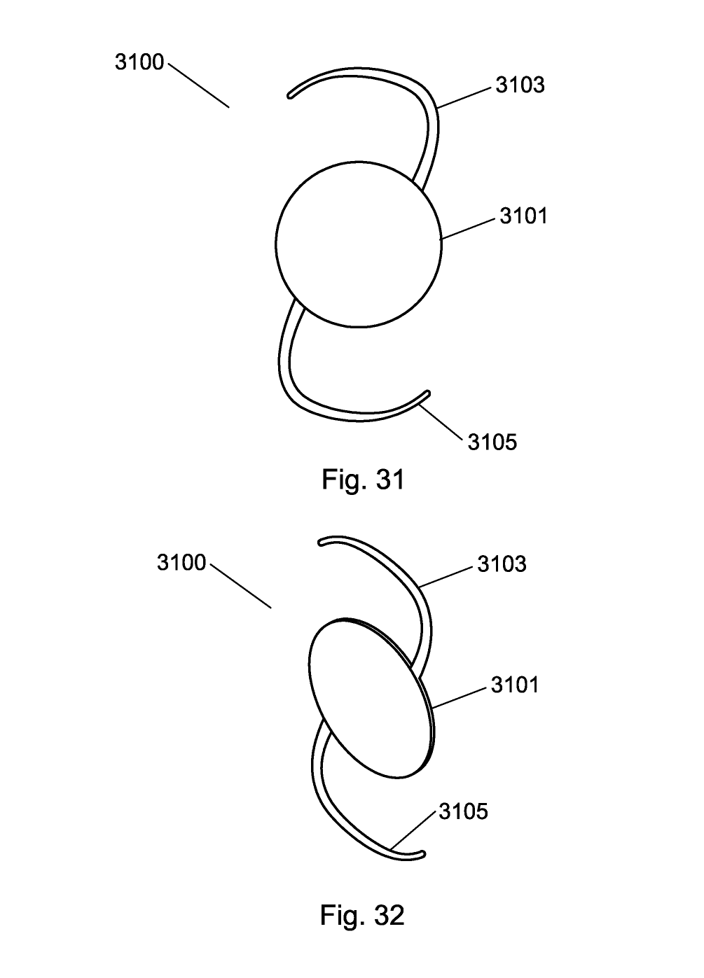

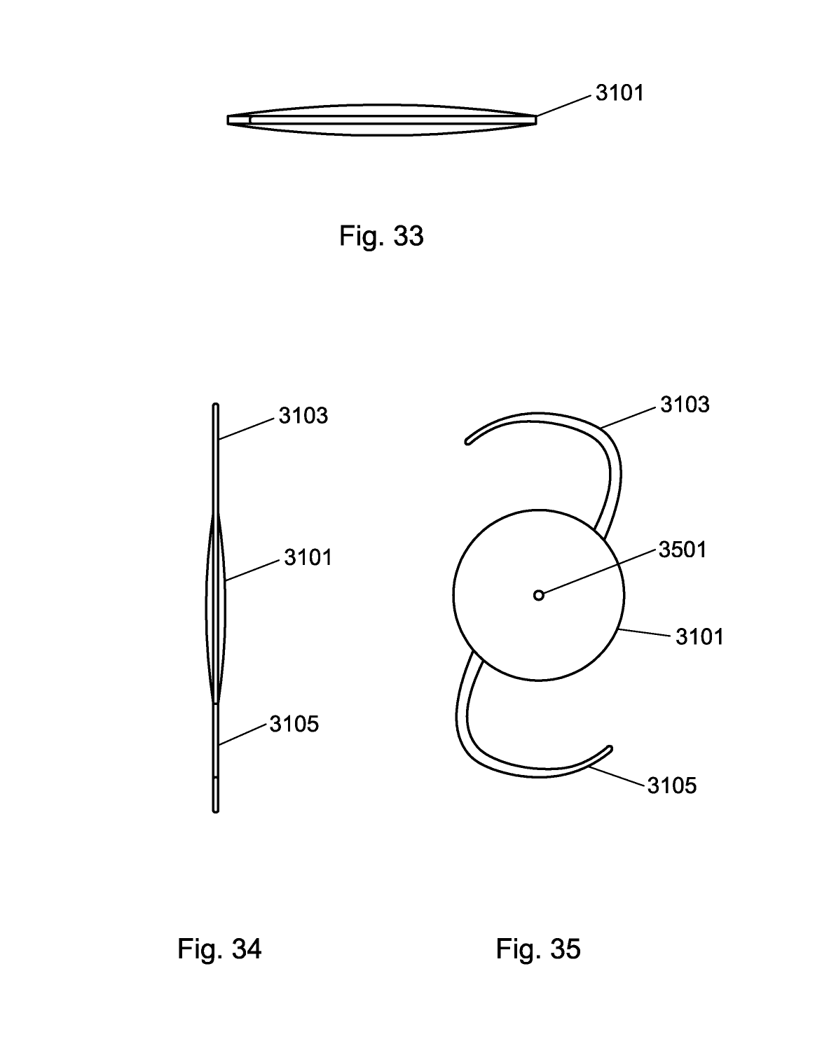

In addition to correction of a cataractous lens, the present invention may be used to correct vision in patients with currently implanted intraocular lenses. FIG. 31 is a plan view of a commonly used intraocular lens 3100. The lens 3101 can be seen along with a first haptic 3103 and a second haptic 3105. FIG. 32 is a perspective view of a commonly used intraocular lens, FIG. 33 is a side view of a commonly used intraocular lens, and FIG. 34 is a rotated side view of a commonly used intraocular lens. These figures are for example only. The intraocular lenses for which this invention can interact and treat problems of presbyopia and refractive error can be of any shape and be anterior or posterior IOL's. They may have plates or haptics. They lenses may be premium, mutifocal, accommodating, or single power lenses.

To deploy the lens system of the present invention in a primary intraocular lens, a small hole or insertion point 3501 must be made in the intraocular lens. The hole may be of a size as small as approximately 0.1 mm. or as large as 3 mm. It is anticipated that the optimal sized hole or insertion point will between 0.25 mm. and 1 mm. The hole could be for the creation of a pinhole effect, and in that case the optimal hole may be 0.18 mm. The hole could be between 0.1 mm. and 2 mm., however. The hole may be centrally located, or off axis, or peripherally located in the optic of the primary IOL. The hole could be round, or another shape. There could be one, two, three or four holes made. The hole may be round, cylindrical, square, rectangular, “T” shaped, oval, linear, or “L” shaped, or any variation thereof. The instrument or device that makes the hole may have a round shape, or cylindrical, square, rectangular. “T” shaped, oval, linear, or “L” shaped, or any variation thereof. The insertion point 3501 may be made with a surgical instrument, a needle, a rotating cutting device such as a drill, laser, diathermy, a heat source, or the like. The insertion point or hole will be made using the surgical tool that enters the eye through the peripheral cornea and can be rotated so the internal opening faces posterior in the eye. The insertion point or hole can be made by the device as described above that can be positioned and manipulated through said surgical instrument. The hole in the primary IOL will be made to specifically coapt or fasten with a protrusion on the novel secondary IOL described as an invention herein. The secondary lens that interacts with the primary IOL may be tubular or of a design described later in the figures section. FIG. 35 is a plan view of a commonly used intraocular lens readied for placement of a secondary intraocular lens of the present invention showing the insertion point 3501 in the lens 3101 of the primary intraocular lens. The insertion point could be round, or another shape. The insertion point may be centrally located, or off the visual axis, or peripherally located in the optic of the primary IOL. There could be one, two, three or four holes made. The insertion point may be round, cylindrical, square, rectangular, “T” shaped, oval, linear, or “L” shaped, or any variation thereof. The instrument or device that makes the hole may have a round shape, or cylindrical, square, rectangular, “T” shaped, oval, linear, or “L” shaped, or any variation thereof. The protrusion of the secondary IOL will fit into the insertion point. The secondary IOL with the protrusion will be guided by the surgeon so that the protrusion can be placed through the insertion point and the two lenses (primary and secondary) will be fastened together to treat presbyopia or to correct refractive error problems in the pseudophakic eye. An optical engineer validates that optical scatter and distortion will be insignificant, minor, unnoticeable, or non-bothersome as pertains to the hole and secondary IOL. An primary IOL with such a hole (whether centrally or off axis) is tested on an optical bench and the pinhole is shown to correct for refractive error and presbyopia. The primary IOL and the fastened secondary IOL with the retention structure is tested and the optical characteristics are favorable for treating presbyopia or correcting refractive error.

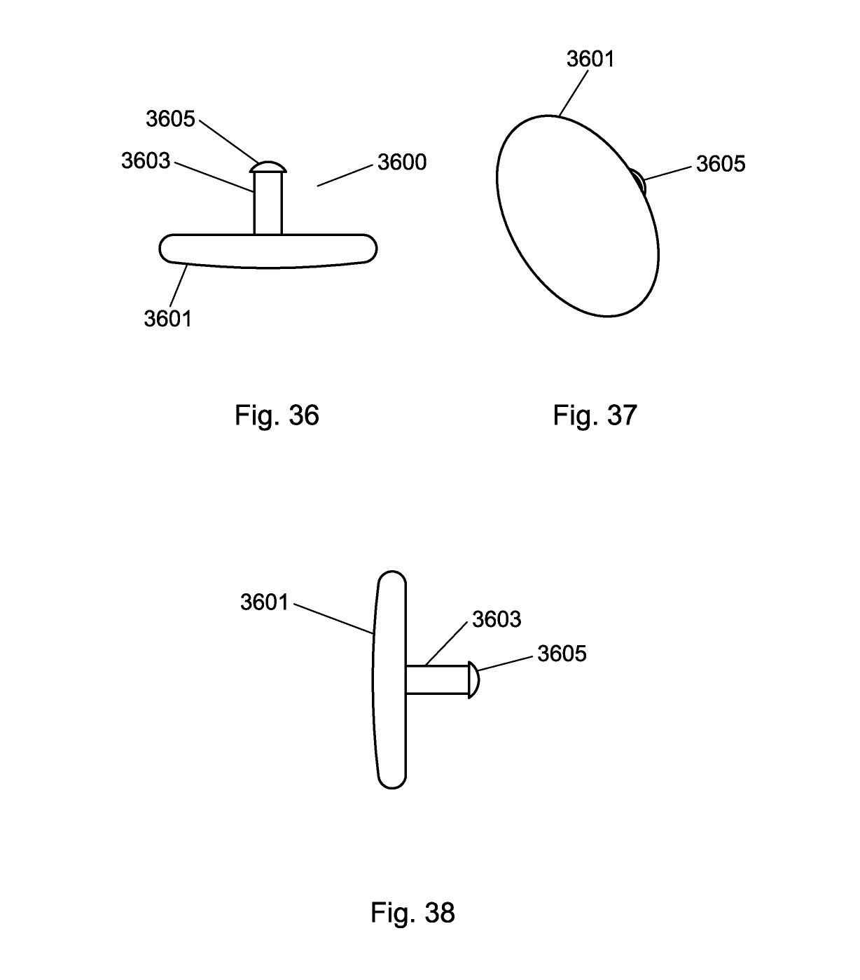

FIGS. 36-46 depict an exemplary embodiment of the secondary intraocular lens that would be placed in the insertion point 3501 of the lens 3101 of the intraocular lens depicted in FIGS. 31-35. The self stabilizing secondary intraocular lens 3600, as seen in the plan view depicted in FIG. 36, has a lens 3601, a retention structure (also described herein as a protrusion) 3603 protruding from, and generally perpendicular to, the novel secondary lens 3601. There may be a head 3605 at the end of the retention structure 3603 that is wider than the protrusion 3603 separated from the lens 3601. The head 3605 serves to retain the self stabilizing secondary intraocular lens in the insertion point 3501 of the intraocular lens. The head 3605 may not be included in some embodiments. Simple expansion of the protrusion such as can be seen with a hydrogel polymer may suffice to fixate and secure the secondary IOL. The protrusion could be round, or another shape. The protrusion 3603 may be centrally located, or off the visual axis, or peripherally located in the optic of the primary IOL. There could be one, two, three or four protrusion 3603 in various embodiments of the invention. The protrusion may be round, cylindrical, square, rectangular, “T” shaped, oval, linear, or “L” shaped, or any variation thereof. In one embodiment the secondary intraocular lens comprises a lens 3601 affixed to a retention structure (or protrusion, these terms can be interchanged) 3603, the retention structure 3603 having a generally cylindrical form and having a first end and a second end, the first end of the retention structure 3603 perpendicularly affixed to the lens 3601, and a head 3605 having a diameter greater than the diameter of the retention structure 3603 and affixed to the second end of the retention structure 3603. In other embodiments, there is no head 3605 on the secondary IOL. In other embodiments of the invention the protrusion or retention structure could be cylindrical or another shape. The protrusion or retention structure 3603 may be centrally located, or off the visual axis, or peripherally located in the optic of the primary IOL. There could be one, two, three or four of these retention structures 3603 on a single lens 3601. In various embodiments of the invention, the protrusion or retention structure may be square, rectangular, “T” shaped, oval, linear, or “L” shaped, or any variation thereof. The key feature of the retention structure that is part of the secondary IOL is that when placed into the hole in the primary IOL it can coapt, fit, fasten and or otherwise remain stable. The size of the optic of lens 3601 will be different depending on the exact problem addressed (presbyopia or refractive error), and could be as small as one or two mm. in diameter. It could be as large as three, four, five, six or seven mm. in diameter. The secondary IOL optic may be extremely thin (less than 0.1 mm. in diameter), or between 0.1 mm. and 1 mm. in diameter. The optic could be as wide as two or three mm. in diameter. This lens may be foldable, rollable, bendable, or expandable or contain combinations of these characteristics. The retention structure or protrusion may be of the same material as the optic, or different. The retention structure or structures may in some embodiments have a stiffness that allows it to be placed through the insertion hole in the primary IOL. The retention structure or structures may in some embodiments have a diameter, or measurements for a shape that is not cylindrical, less the insertion hole that was made in the primary IOL.

The secondary IOL in the various embodiments described herein may be multifocal or accommodating in nature, as well as custom or toric. Toric indicating it can correct for astigmatism. A marking on the secondary IOL may allow for proper alignment in the eye.

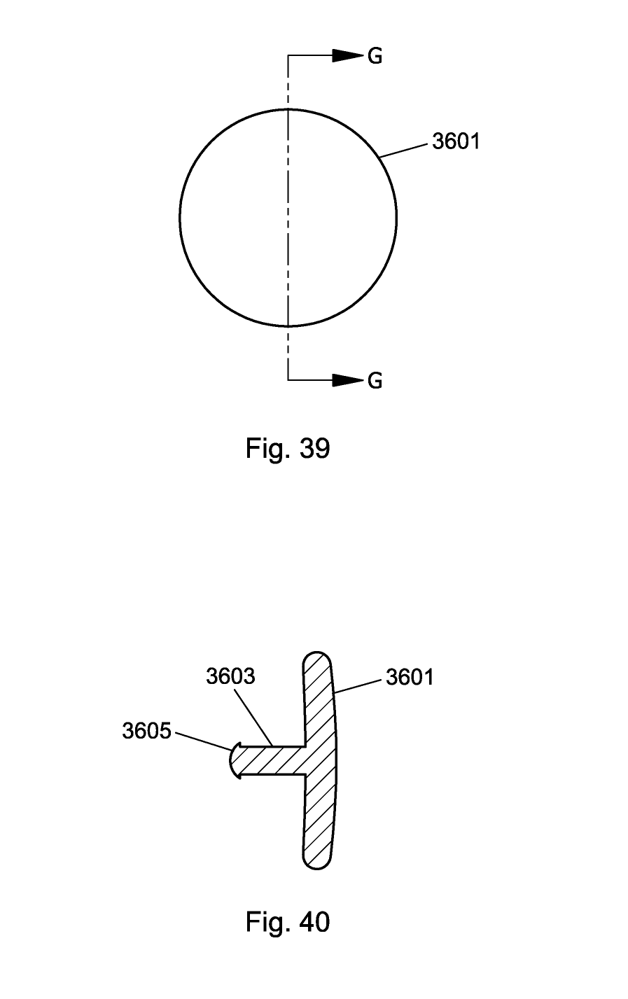

FIG. 37 is a perspective view of the secondary intraocular lens of the present invention. FIG. 38 is a rotated side plan view of the secondary intraocular lens of the present invention. FIG. 39 is a top plan view of the secondary intraocular lens of the present invention. FIG. 40 is a side plan view of the secondary intraocular lens of the present invention cut along line G-G of FIG. 39. In FIGS. 36-39, the lens 3101 is depicted fully open and unfolded, as it would be once deployed in an intraocular lens. The protrusion or retention structure 3603 may be centrally located, or off the visual axis, or peripherally located in the optic of the primary IOL. There could be one, two, three or four of these retention structures 3603 on a single lens 3601. In various embodiments of the invention, the protrusion or retention structure may be square, rectangular. “T” shaped, oval, linear, or “L” shaped, or any variation thereof. The key feature of the retention structure that is part of the secondary IOL is that when placed into the hole in the primary IOL it can coapt, fit, fasten and or otherwise remain stable. The size of the optic of lens 3601 will be different depending on the exact problem being addressed (presbyopia or refractive error), and could be as small as one or two mm. in diameter. It could be as large as three, four, five, six or seven mm. in diameter. The secondary IOL optic may be extremely thin (less than 0.1 mm. in diameter), or between 0.1 mm. and 1 mm. in diameter. The optic could be as wide as two or three mm. in diameter. This lens may be foldable, rollable, bendable, or expandable or contain combinations of these characteristics. The retention structure or protrusion may be of the same material as the optic, or of a different material. The retention structure or structures may in some embodiments have a stiffness that allows it to be placed through the insertion hole in the primary IOL. The retention structure or structures may in some embodiments have a diameter, or measurements for a shape that is not cylindrical, less the insertion hole that was made in the primary IOL. To facilitate deployment of the self stabilizing secondary intraocular lens, the lens 3101 is folded or otherwise compacted to allow deployment through the delivery conduit of the surgical tool of the present invention. The self stabilizing secondary intraocular lens may be made from a hydrophilic material. Suitable hydrophilic materials may include, but are not limited to, Hema (Poly-hydroxy-ethylene thacrylate), 2-hydroxyethyl methacrylate (HEMA), hydrophilic acrylic, 2 HEMA hydrophilic acrylic, hydrogels, hydrophilic polymers, fluorocarbon-sulfone. Any transparent and biocompatible material from which a contact lens or intraocular lens can be made is embodied in the lens designs and materials herein. Hydrophobic acrylic or silicone lenses may also be used.

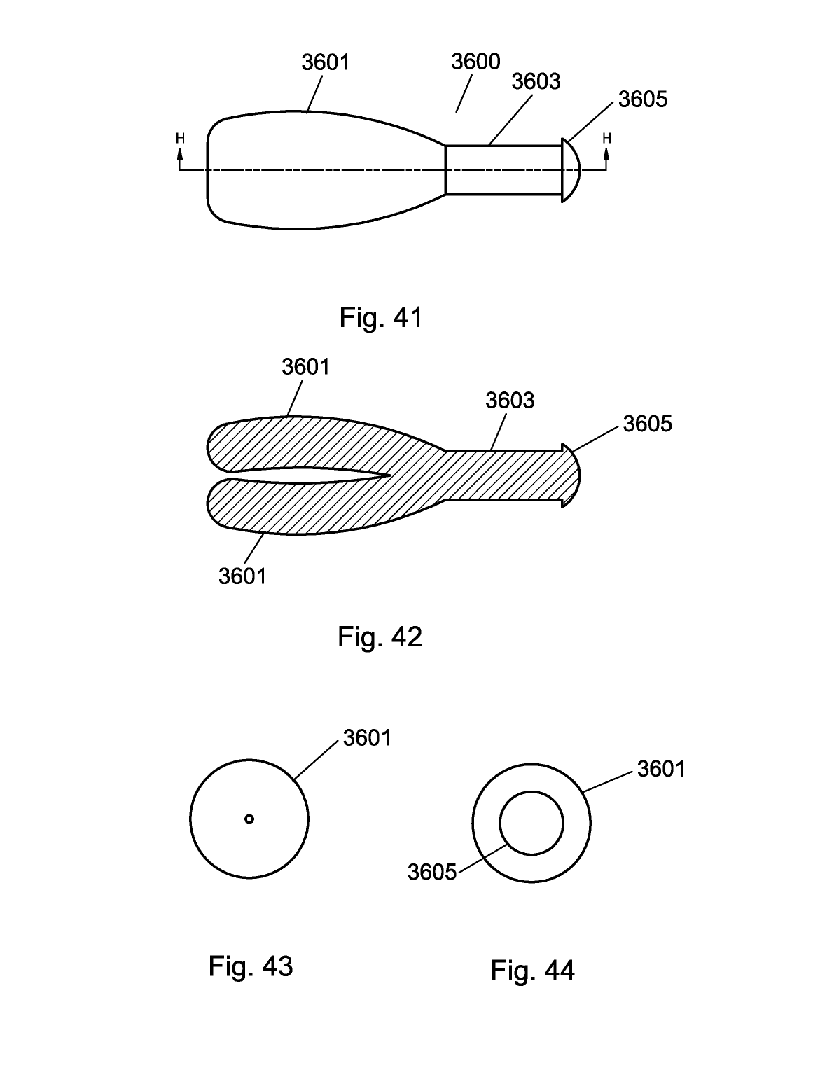

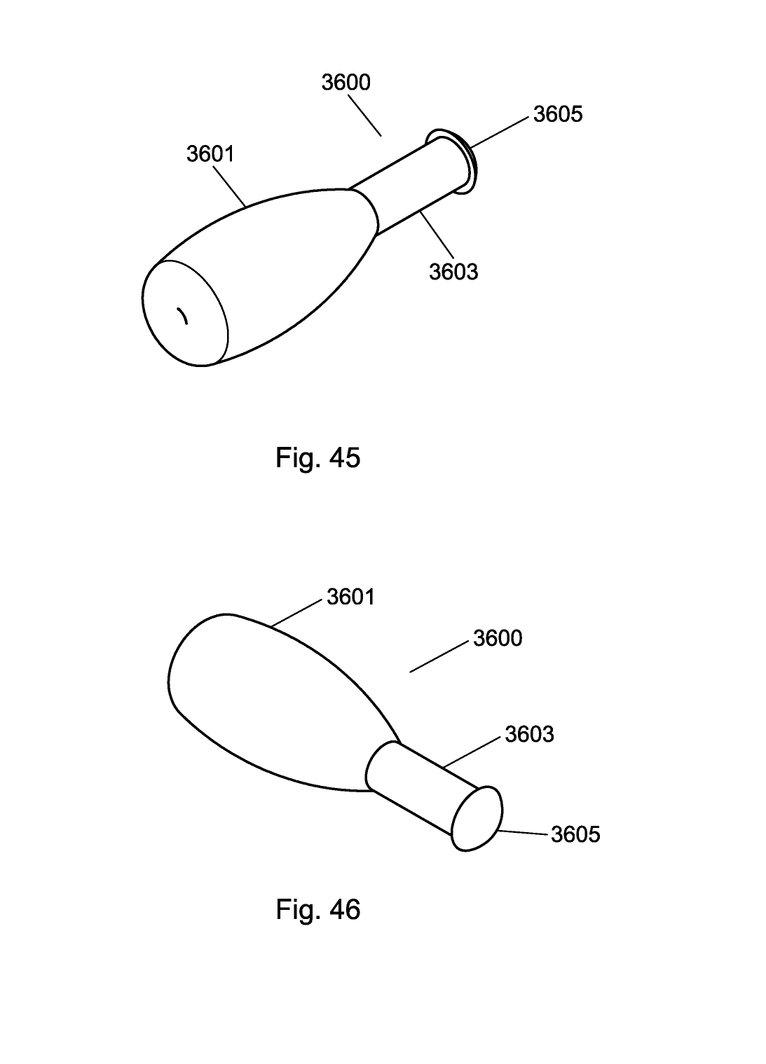

FIG. 41 is a side plan view of the secondary intraocular lens of the present invention in a folded position showing the compacted and ready to deploy secondary intraocular lens. 3605 need not be present. The overall shape would be somewhat different if the retention structure or protrusion is not centered or is of a different shape as described above, however, the general idea is presented thoroughly so that the secondary IOL with the retention structure may be folded or generally altered or compressed so as to go through the conduit in the surgical instrument related to this invention for placement in secure attachment to the primary IOL. Similar alterations and clarifying comments regarding the other embodiments, part of and contained fully in the invention herein, apply also to the following figures. FIG. 42 is a side plan view of the secondary intraocular lens of the present invention in a folded position cut along line H-H of FIG. 41. FIG. 43 is a bottom plan view of the secondary intraocular lens of the present invention in a folded position. The lens may be rolled or otherwise manipulated to fit through the insertion system, folding is only one embodiment. FIG. 44 is a top plan view of the secondary intraocular lens of the present invention in a folded position. FIG. 45 is a perspective view of the secondary intraocular lens of the present invention in a folded position. FIG. 46 is a rotated perspective view of the secondary intraocular lens of the present invention in a folded position. Insertion point and hole may in some embodiments be used interchangeably in this document. These figures are for example only and do not limit the secondary IOL's shape when readied for insertion in any way. The protrusion may be smaller or larger, the novel IOL may be thinner, shorter, thicker, wider, longer or off center when placed though the insertion surgical instrument. Key beneficial aspects of this part of the inventions: a secondary IOL with a protrusion or retention structure and a novel surgical tool for the creation of an insertion hole for the secondary IOL into a primary IOL and the surgical tool that can also be used for placement of the secondary lens, is that the process and devices allow for the placement of a secondary IOL in the visual axis, which has optical advantages.

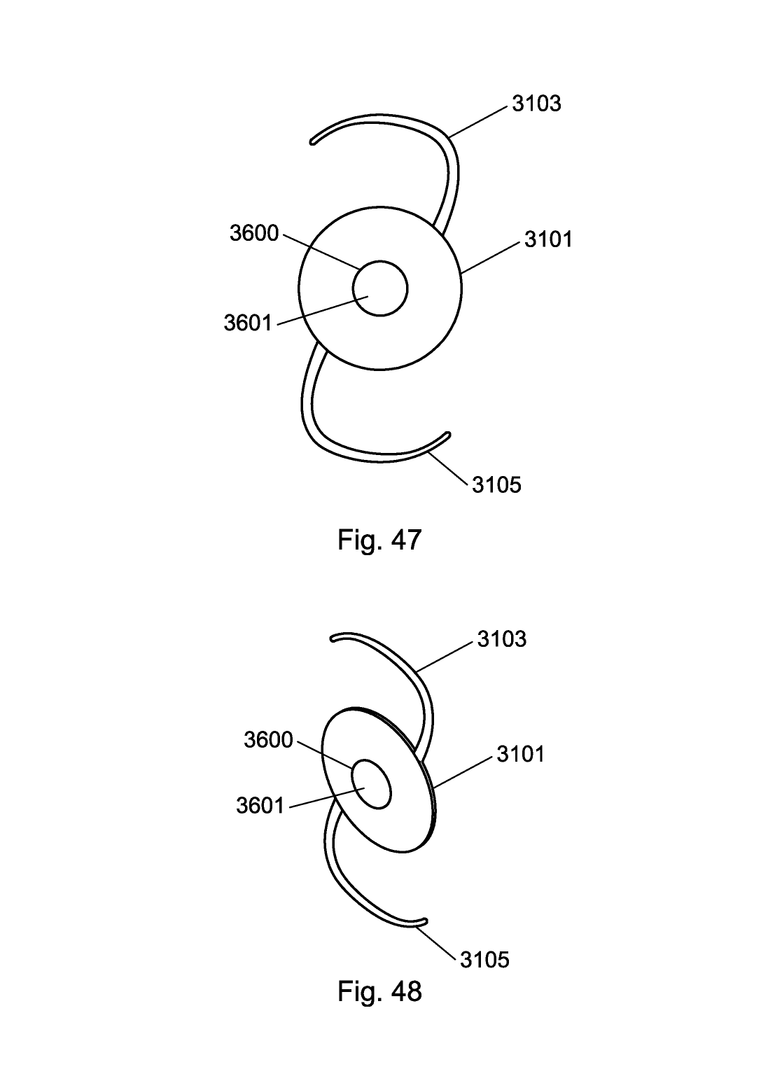

FIG. 47 is a plan view of a commonly used intraocular lens with the secondary intraocular lens installed therein. The secondary intraocular lens is delivered through the delivery conduit of the surgical tool 100 such that the retention structure 3603 (see FIG. 36) is pushed through an insertion point 3501 (see FIG. 35) such that the head 3605 (not required, see FIG. 36) is pushed through the insertion point 3501 in a compacted state and then will expand once the head clears the insertion point 3501 and is free to expand. The optic of the secondary IOL 3601 may be centrally located on the visual axis, or off axis in some situations. The size of the optic of lens 3601 will be different depending on the exact problem addressed (presbyopia or refractive error), and could be as small as one or two mm. in diameter. It could be as large as three, four, five, six or seven mm. in diameter. The secondary IOL optic may be extremely thin (less than 0.1 mm., in diameter), or between 0.1 mm. and 1 mm. in diameter. The optic could be as wide as two or three mm. in diameter.

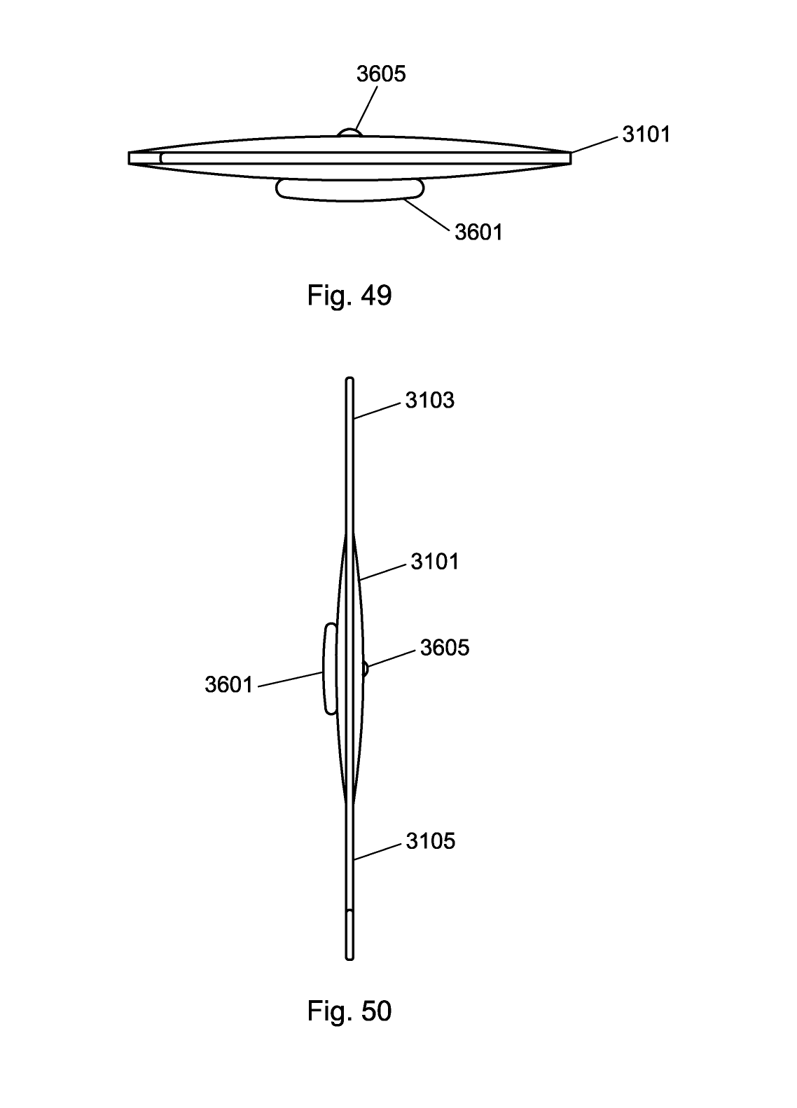

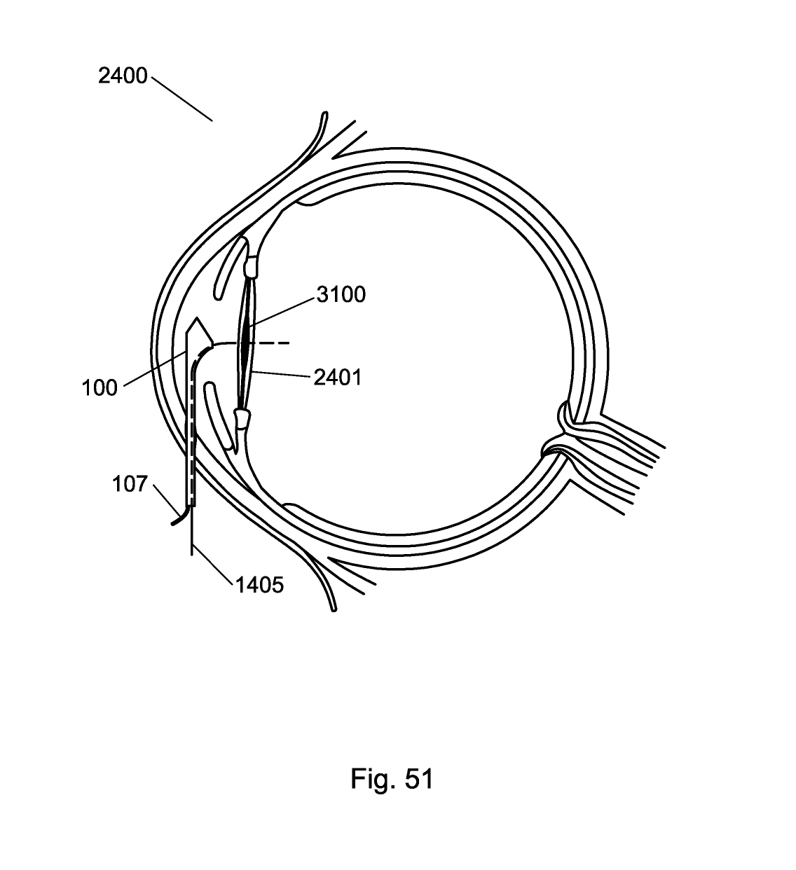

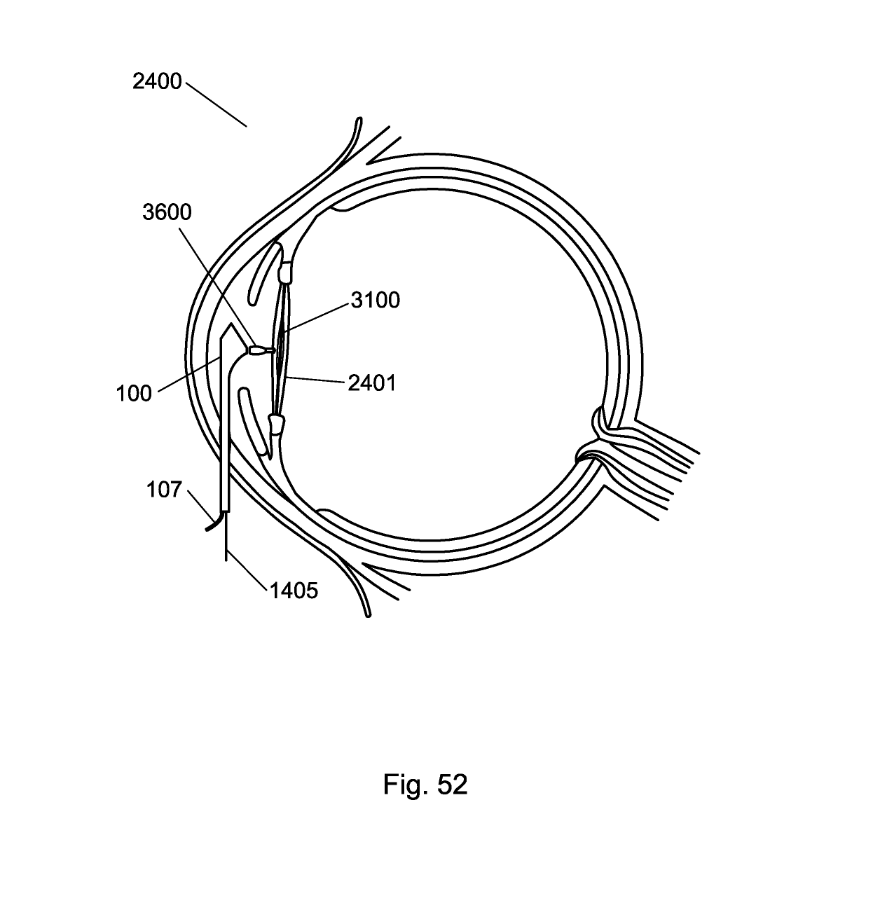

FIG. 48 is a perspective view of a commonly used intraocular lens with the secondary intraocular lens placed in position. FIG. 49 is a side plan view of a commonly used intraocular lens with the secondary intraocular lens installed therein. FIG. 50 is a rotated side plan view of a commonly used intraocular lens with the secondary intraocular lens installed therein. The protrusion or retention structure 3603 (not visible) may be centrally located, or off the visual axis, or peripherally located in the optic of the primary IOL. The head 3605 may be indistinguishable from the form factor of the retention structure, or may be larger in diameter. In any case, the secondary IOL is held in place by the retention structure, and the optic 3601 is in or near to the visual axis of the eye and presbyopia and or refractive errors in a pseudophake can be treated.

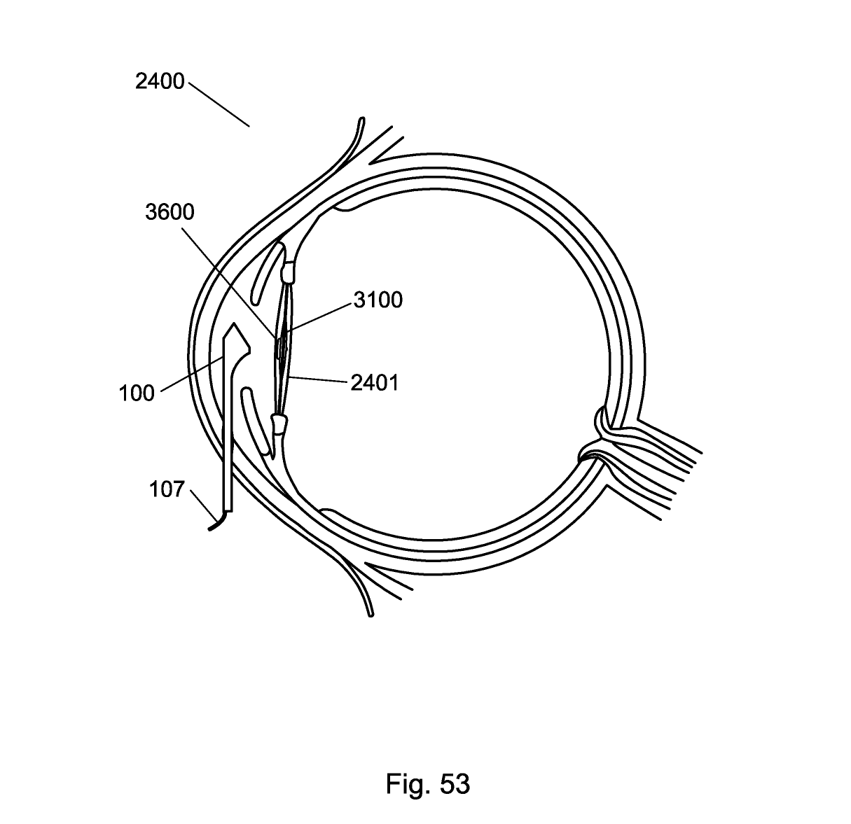

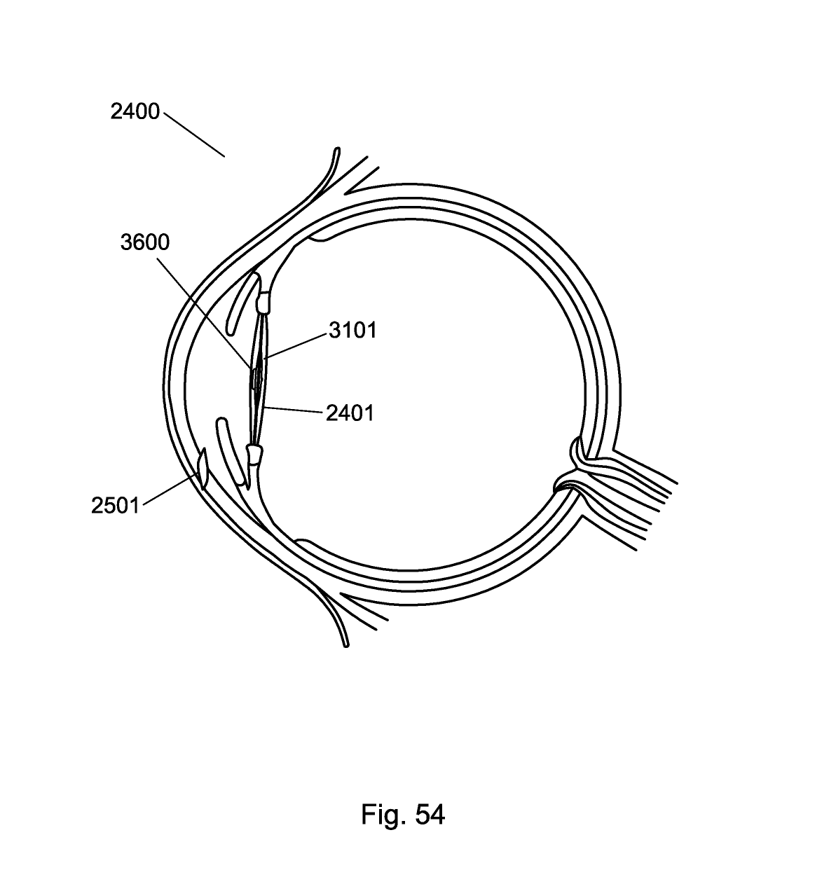

FIGS. 51-54 depict a typical installation of the self stabilizing secondary intraocular lens of the present invention. As depicted in FIG. 35, an insertion point 3501 is made in an intraocular lens. FIG. 51 depicts the insertion point being made in an intraocular lens 3100 with a guide wire 1405, although other techniques may also be used. Other techniques include, but are not limited to heat, laser energy, diathermy, or rotational or drilling energy. The intraocular lens 3100 has been previously placed in the capsular bag of the lens 2401 depicted in FIG. 51. The IOL 3100, however, can be in the anterior chamber or posterior chamber. It can be in the capsule, or in the sulcus. It can be sutured or secured by haptics as is traditional. Once the insertion point 3501 is made, the self stabilizing secondary intraocular lens is pushed into the insertion point 3501 by way of force applied to the guide wire 1405 or related guiding structure, as can be seen in FIG. 52. As previously depicted in FIGS. 36-46, the retention structure 3603 is placed through the insertion point 3501 until insertion is stopped by the lens 3601 itself. The protrusion or retention structure 3603 may be centrally located, or off the visual axis, or peripherally located in the optic of the secondary or primary IOL. There could be one, two, three or four of these retention structures 3603 on a single lens 3601. In various embodiments of the invention, the protrusion or retention structure may be square, rectangular, “T” shaped, oval, linear, or “L” shaped, or any variation thereof. The key feature of the retention structure that is part of the secondary IOL is that when placed into the hole in the primary IOL it can coapt, fit, fasten and or otherwise remain stable. The guide wire 1405 is then retracted, as can be seen in FIG. 53. The head 3605 that has been deformably placed through the insertion point 3501 is now free to expand or fasten, and serves to retain the self stabilizing secondary intraocular lens 3600 in the intraocular lens 3100. The surgical tool 100 is utilized in a manner similar to that described by way of FIGS. 24-30. Lastly, the surgical tool 100 is removed, and the self stabilizing secondary intraocular lens is free to expand and become secure in the intraocular lens. FIG. 54 shows an exemplary secondary IOL in position and attached to the primary IOL.

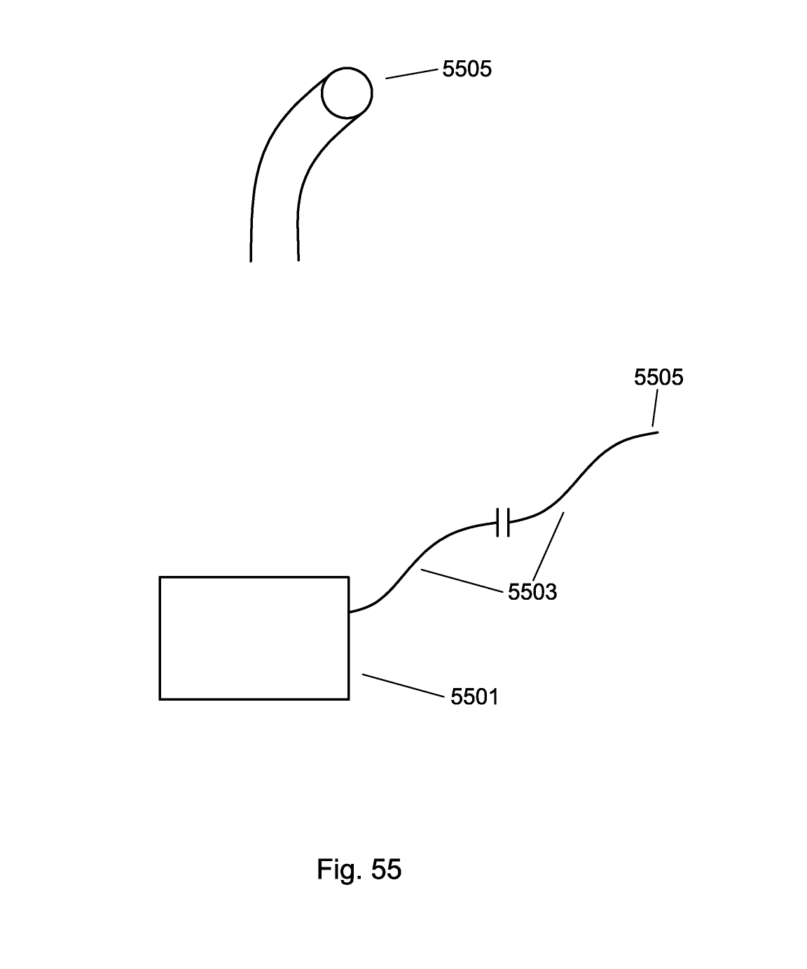

FIG. 55 shows an energy source 5501 (similar to 3701) connected to a cable, wire, or guiding device similar to 5503 (similar to 3702) with an end, tip or insertion hole creation device 5505 (similar to 3703). In the top image, 5505 is shown enlarged so an example round shape is shown. The cable, wire, or guiding device may convey mechanical, electrical, photonic, laser, heat signals or energy. 5503 may be of various lengths, thus the gap can represent any of various lengths. The length of 5505 may vary from several centimeters to a meter or longer, depending on the configuration chosen by surgeon and/or manufacturer. 5503 may convey instructions to a micromachine in some embodiments. 5503 is attached to an energy source capable of creating, for example, heat, diathermy, cautery, laser, drilling, or cutting at the tip 5505. The tip 5505 and the cable, wire, or guiding device can be, for example, slidably disposed in the surgical tool described in FIGS. 1 to 14, with emphasis on FIG. 14. The tip and cable, wire, or guiding device can be advanced once the novel surgical tool is in position and the tip can then engage the primary IOL to make the insertion hole. The combination of 5501, 5503, and 5505 may be used in concert with an advancing secondary IOL. In this FIG. 55, 5503 is round and creates a round insertion hole in the primary IOL. In some embodiments this is required and planned, but 5505 may also have other shapes such that when placed in contact with a primary IOL the shape will create a correlated shaped insertion point (not round in those cases). Heat could do that well for example, to create an oval, square, rectangle, “L” shape, “T” shape, or “+” shape.

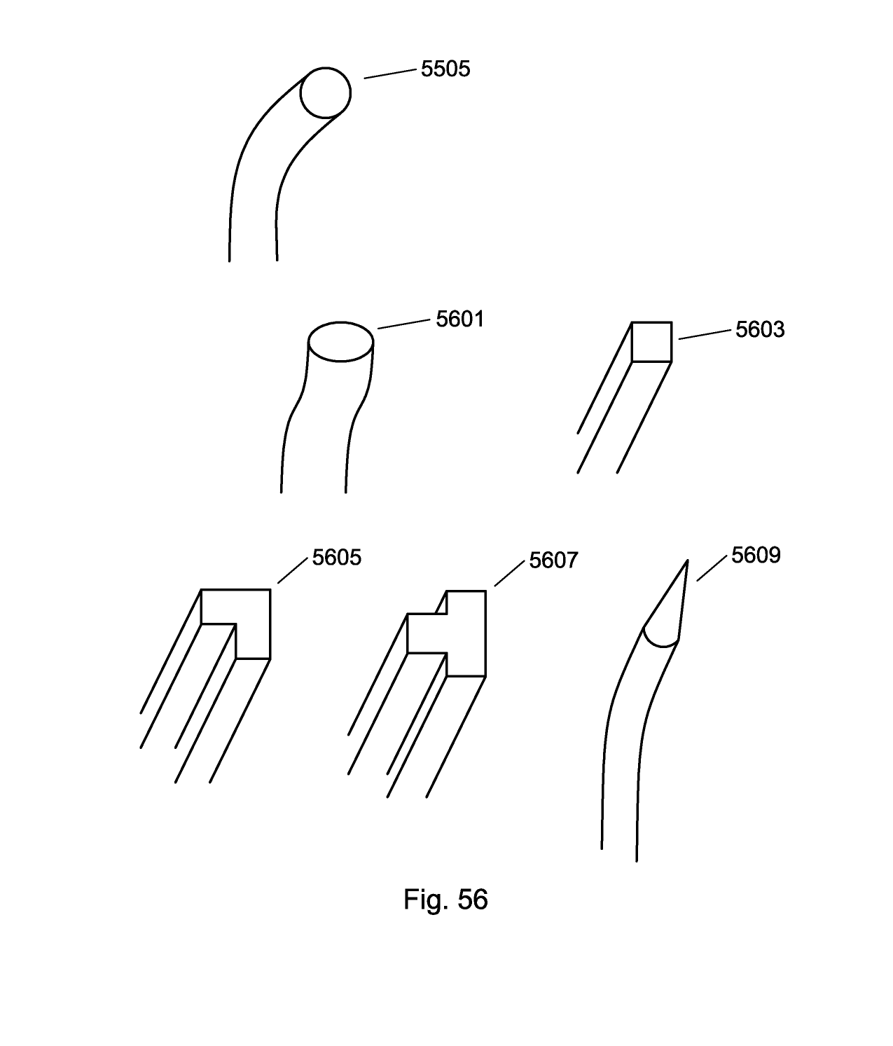

By way of example, FIG. 56 shows the tip of the energy source again, the insertion hole creation device, 5505, as round. When labeled as 5601 the same tip is shown oval, 5603 the tip is square or rectangular, 5605 the tip is “L” shaped, and 5605 shows the tip with a “T” shape. Other shapes constitute parts of this invention as well. The reason a shape other than round may be selected for the insertion hole creation device tip is that such a different shape could limit rotation of the secondary IOL, which has a protrusion or retention structure that would match the insertion hole. By limiting rotation control of lens alignment, one can ensure the best possible optical performance of the secondary IOL, especially if it is a custom lens or a toric lens. 5609 shows that the tip may be tapered, and narrower at the most distal end compared to the aspect of the device that makes the greatest size insertion point. The tip of the device could create a hot point to melt the primary IOL, or it could use electrical energy, cautery, diathermy, radiofrequency energy, light, laser, drilling, or a micromachine to pierce the primary IOL and with minimal force create the hole required for insertion of the retention structure of the secondary IOL. The secondary IOL may have a retention structure that mimics or is similar or slightly smaller than the shapes discussed herein.

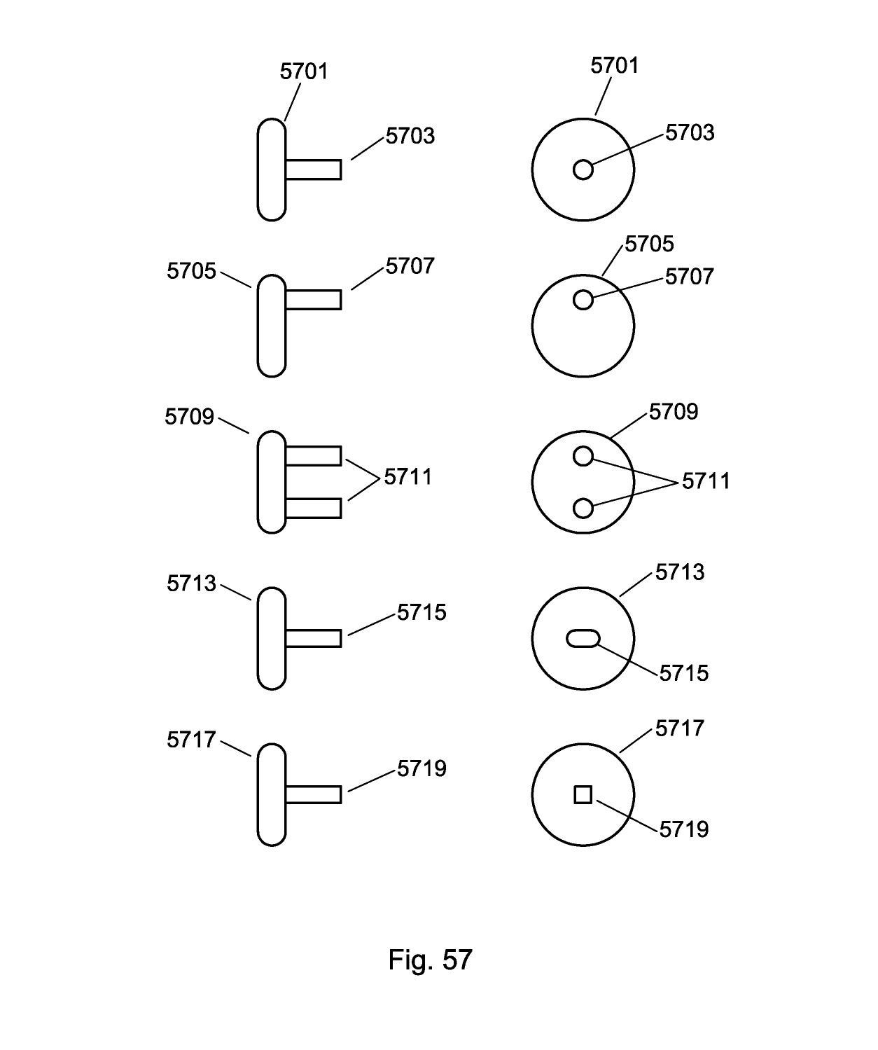

FIG. 57 shows cross section and top plan views of the optic previously also represented as 3601, here represented as 5701, 5705, 5709, 5713, and 5717 along with the retention structure or protrusion previously also represented as 3603, here represented as 5703, 5707, 5711, 5715, and 5719. As seen here, there is no cap 3605. There may or may not be a cap like 3605 in these and other secondary lens designs. These views show that the retention structure 5703, 5707, 5711, 5715, and 5719 may be centered or off center or may be singular or multiple. The retention structure 5703, 5707, 5711, 5715, and 5719 may have a shape that is round, oval, oblong, square, rectangular, or other shape and that shape may correlate closely with the shape of the insertion hole in the primary IOL. It will also correlate with the tip of the energy source which makes the insertion hole in the primary IOL. These components are integrated as a system in one embodiment of this invention. As can be interpreted easily, the drawings of FIG. 57 can be further repeated with a multiple of different shapes, with or without an end cap 3605, such that the drawings could show another shape for the retention structure. The length of the retention structure from the back surface of the secondary IOL can be from 0.25 mm. to 3 mm., but could be longer or shorter. The diameter or greatest width of a round or non-round shape, respectively, can be between 0.05 mm. and 2 mm. The end cap 3605 if utilized in an embodiment could be slightly larger than the diameter of the round retention structure, or width of the shape generally if it is not round. There can be multiple differently shaped retention structures as well which can be central or peripheral to the center of the secondary IOL. The retention structure may protrude perpendicular or close to perpendicular from the back surface of the lens, but it also may protrude at an angle different than close to 90 degrees. Measured from its most acute point, the angle may be as little as 15 degrees. One reason an angle less than 90 degrees measured from its most acute point may be helpful is to limit reflections.

The power of the secondary IOL with the protrusion described herein can range from −20 diopters to +20 diopters. In general the power of the secondary IOL will be in the range of −5 to +5 diopters. The optic of the secondary IOL may also be toric and may be able to correct for astigmatism. Thus, refractive errors described herein can include astigmatism for which some embodiments of this lens can correct. The fastening aspect of the present invention allows for a secure method to hold a toric lens in place. Thus, a toric secondary IOL with a protrusion is also encompassed in the present invention descriptions.

Another key aspect of this invention is the placement of a custom secondary IOL in front of a primary IOL. The invention includes the placement of single power, premium (multifocal or accommodating), presbyopic corrective, and toric secondary IOL's. The secondary IOL of the present invention may be aspheric. These lenses can typically correct lower order aberrations such as negative defocus (myopia), positive defocus (hyperopia) and regular astigmatism, and presbyopia. When referring to custom the intent also includes lenses manufactured specifically for a patients residual higher order aberrations. Wave front technology, corneal and/or other aberration detecting methods may be used to identify, detect, measure, or evaluate higher order aberrations. Higher order aberrations include but are not limited to spherical aberration, coma, and trefoil. Measuring wavefront and refraction in an eye can be used to generate a custom secondary IOL for the present invention. Shack-Hartman, Tschering systems, ray tracings, Skiascopy, and other types of aberrometers are some of the methods that may be used for detecting the pseudophakic eye's aberrations. The method for the detection of aberrations is not limited to these tests for determining the custom secondary IOL of the present invention. RMS, Zernike polynomials, and other methods may be used to quantify aberrations when planning for a custom secondary IOL of the present invention. The information gathered about aberrations of the subjects eye can then be used to custom manufacture lenses that correct for these aberrations using lathe cutting, laser treatment following initial manufacture, molding and casting processes, and three dimensional printing to manufacture the custom secondary IOL. Another aspect of the invention is the use of three dimensional printing to create, test, and manufacture custom IOL's not only IOL's of the present discussion herein (secondary IOL with a protrusion or retention structure), but any primary, secondary, or tubular IOL. The secondary lens of the present invention may be a gradient index lens (GRIN).

For further detailed specific elaboration as well as review, the novel technologies and inventions of the present invention include, but are not limited to:

Another embodiment of the present invention includes an intraocular lens for insertion through a hollow instrument with anchoring protrusions extending radially and posteriorly.

Another embodiment of the present invention includes an intraocular lens with anchoring protrusions that secure into an eyelet on a haptic of a primary IOL.

Another embodiment of the present invention includes an intraocular lens with anchoring protrusions that secure into a small hole in the capsule. The hole in the capsule described above is made by a laser or a tool (the tool is a sharp device or applies energy such as heat), the tool is advanced through an instrument into the eye to make the small capsulotomy holes.

An embodiment of the present invention includes a combination lens system of an IOL with eyelet on haptic and secondary IOL with a protrusion that secures to the eyelet. The protrusion from the optic in the present invention can be used to engage the capsule of the lens.

The protrusion protrudes at different angles depending on the embodiment. The protrusion protrudes horizontal to the optic in one embodiment. The protrusion protrudes at an angle toward the posterior surface of the optic in one embodiment.
An embodiment of the present invention includes a protrusion on the optic for engaging and manipulating the secondary IOL.
An embodiment of the present invention includes the use of laser energy delivered through a fiberoptic cable to create a hole in the capsule.

The blade on the surgical instrument described herein is retractable in some embodiments. The blade can be retracted by engaging an attachment on the instrument that can be manipulated by the surgeon to retract the blade. By retractable it is meant the blade can be withdrawn from its most outward position relative to the instrument generally a distance of between 1 mm. and 10 mm. depending on the embodiment. By retractable it is meant the blade can be withdrawn from its most outward position relative to the instrument generally at distance of between 2 mm. and 4 mm. depending on the embodiment. A connection including those of wire metal or plastic which connects to the blade and an activator is used to manipulate the blade from its outward position; it is pulled or retracted back toward the surgeon. The retractable blade is, in some embodiments, is triggered to retract by the surgeon's activation of the release mechanism.

An apparatus and method for treating a pseudophakic eye, comprising: a surgical tool formed with or without an angle to allow entry of the surgical tool through the peripheral cornea of the eye. If there is an angle, the angle is formed by the convergence of a shaft for surgical handling and an operative head; the surgical tool is used to create an insertion hole in the primary intraocular lens.

The surgical instrument described, in some embodiments, has a straight shaft. Various embodiments have slight angles of 10 degrees or less. In some embodiments, the surgical insertion instrument has a bend. In some embodiments it is straight.

An apparatus and method for treating a pseudophakic eye, comprising: a surgical tool formed with an angle to allow entry of the surgical tool through the peripheral cornea of the eye where the angle is formed by the convergence of a shaft for surgical handling and an operative head; the surgical tool is used to place a secondary IOL in piggyback fashion into the eye.

An apparatus and method for treating a pseudophakic eye, comprising: a surgical tool formed with an angle to allow entry of the surgical tool through the peripheral cornea of the eye where the angle is formed by the convergence of a shaft for surgical handling and an operative head; the surgical tool is used both to create an insertion hole in the primary intraocular lens and for the placement a secondary IOL in piggyback fashion into the eye.

An apparatus and method for treating a pseudophakic eye, comprising: a surgical tool formed with an angle to allow entry of the surgical tool through the peripheral cornea of the eye where the angle is formed by the convergence of a shaft for surgical handling and an operative head; the surgical tool is used to maintain the anterior chamber, to create an insertion hole in the primary intraocular lens and for the placement of a secondary IOL in piggyback fashion into the eye.

An intraocular treatment for a presbyopic pseudophakic eye.

Treatment for a pseudophakic eye with refractive error such as a wrong power primary IOL, myopia, hyperopia, astigmatism, spherical, coma, trefoil, and/or higher order aberrations.

A surgical tool for intraocular surgery that has 1, 2, 3 or 4 conduits. The conduits can be used for the passage of instruments such as guiding structures with an energy source and ability to create an insertion hole in a primary IOL, for passing a secondary IOL, for manipulating a secondary IOL, and for infusion and anterior chamber maintenance as needed.

The surgical tool may have a conduit for an infusion fluid.

The surgical tool may have on the operative head a sharp tool for penetration of a peripheral cornea of the eye. This sharp tool could be similar to a keratotomy knife. It could be, for example, a steel or diamond blade. The sharp tool may be detachable. Detachable blades used with the surgical tool are one embodiment of the present invention.

The surgical tool may have a delivery conduit disposed within said surgical tool for delivery of a device with an energy source that can be used to make an insertion hole in the primary IOL.

The surgical tool discussed herein may have a delivery conduit disposed within said surgical tool for delivery of a secondary intraocular lens system device to coapt or attach via a hole in a primary intraocular lens; and also may have a guiding structure attached to the lens system device and further disposed within the surgical tool.

A surgical apparatus that is linear in part and can be placed through a surgical instrument and that has an energy source proximal such that the tip of the device can create an insertion hole in a primary IOL. The distal tip may be pointed (such as with a taper) and once the tip widens the tool may have a shape that is round, oval, square, rectangular, “L” shaped, “T” shaped. “+” shaped, or have some other shape amenable to attaching and fixating a secondary IOL with a protrusion or retention structure.

The energy source supplied to the tip of the surgical apparatus may include heat, laser, diathermy, cautery, drilling, rotational energy, piercing, and light. The energy source may be attached to the tip of the secondary IOL's protrusion such that in one step the primary IOL has an insertion hole placed and the secondary IOL is attached. Such a step will only minimally or not at all traumatize the primary IOL, which is a key aspect of some embodiments of the present invention.

A surgical device embodied in the present invention can be used to create a pinhole in a primary IOL in a pseudophakic eye.

A surgical device that can be used to enter the eye through the peripheral cornea and can be used to create an insertion hole in a pseudophakic eye and deliver a secondary IOL into the eye so that the secondary IOL can be attached to the primary IOL is also an embodiment of the present invention.

Regarding more information on lenses, a secondary intraocular lens with a protrusion or retention structure that is used to attach the secondary IOL to a primary IOL is an embodiment of the present invention.

The secondary intraocular lens of the present invention may be used to treat a pseudophakic eye with a wrong power primary IOL, myopia, hyperopia, astigmatism or higher order aberrations.

The secondary intraocular lens may be toric, single power, custom, accommodating or multi-focal.

The lenses described herein may have a power of between +30 and −30 diopters.

The lens made at least in part with an expandable hydrogel is one embodiment of the present invention.

The lens may be manufactured by casting, molding, injection molding, lathe cutting, and laser ablation of a polymer, and three dimensional printing to correct for higher order aberrations as well as lower order aberrations in some embodiments. The lens may be foldable, rollable, compressible, expandable, shrinkable, flexible, and/or manipulatable through a conduit. Regarding parts, the lens may be one piece, two pieces, three pieces or more. The materials of these parts may be the same or different.

The lens may have a component that is rigid, dehydrated, partially flexible particularly the retention structure so it can be placed through the insertion hole in the primary IOL with guidance in some embodiments of the present invention.

Described herein is a secondary intraocular lens comprising: a lens affixed to a retention structure, the lens is generally round, the retention structure protrudes perpendicular to the lens; the retention structure fits into an insertion space in a primary IOL. The lens may not be perfectly round. The retention structure may protrude at an angle for various reasons including minimizing reflections. The lens may have a retention structure of various shapes already mentioned. The retention structure may be single or multiple. The retention structure may be centrally located or off the central axis. The retention structure may allow for precise alignment of the secondary lens so higher order aberrations and astigmatism can be corrected. The protrusion may be specifically designed to mimic in shape and be similar in size (it may be slightly smaller in some regards) to the insertion hole in the primary IOL, and thus the instrument tip that creates the hole.

Additionally, the present invention includes an apparatus and method for treating a cataractous eye, comprising: a surgical tool formed with an angle to allow entry of the surgical tool through the peripheral cornea of the eye where the angle is formed by the convergence of a shaft for surgical handling and an operative head; the surgical tool is used to create a cylindrical space in the cataract.

The apparatus and method for treating a cataractous eye may comprise a surgical tool formed with an angle to allow entry of the surgical tool through the peripheral cornea of the eye where the angle is formed by the convergence of a shaft for surgical handling and an operative head; the surgical tool is used to place a tubular IOL through the cataract.

The apparatus and method for treating a pseudophakic may comprise a surgical tool formed with an angle to allow entry of the surgical tool through the peripheral cornea of the eye where the angle is formed by the convergence of a shaft for surgical handling and an operative head; the surgical tool is used both to create a cylindrical space through the cataract and for the placement of a tubular lens through the cataract.

The apparatus and method for treating a cataractous eye may comprise a surgical tool formed with an angle to allow entry of the surgical tool through the peripheral cornea of the eye where the angle is formed by the convergence of a shaft for surgical handling and an operative head; the surgical tool is used to maintain the anterior chamber, to create cylindrical space in the cataract and for the placement of a tubular lens through the cataract.

The size of the tool or tools described herein may be appropriate for humans, human infants, human children, domestic small animals, and large animals such as horses; the instrument itself may range from a few mm. long to 50 mm. long to 0.5 meters long.

A tubular lens is described that can expand and provide image quality better than 20/200 and a field of view larger than 5 degrees when placed through a cataract or a primary IOL. In some embodiments the field of view may be between 5 degrees and 180 degrees. The main embodiment has a field of view of 20 to 30 degrees. The tubular lens may provide an acuity of 20/20, or somewhere between 20/20 and 20/100.

An apparatus for treating presbyopia of a pseudophakic eye is described that may comprise a surgical tool formed with an angle to allow entry of the surgical tool through the peripheral cornea of the eye where the angle is formed by the convergence of a shaft for surgical handling and an operative head for surgical placement of a secondary intraocular lens; the operative head having a sharp tool for penetration of a peripheral cornea of the eye; a delivery conduit disposed within said surgical tool for delivery of the secondary intraocular lens system device to a hole in a primary intraocular lens; and a guiding structure attached to the lens system device and further disposed within the surgical tool.

A secondary intraocular lens is described herein comprising a lens affixed to a retention structure; the retention structure having a generally cylindrical form and having a first end and a second end; the first end of the retention structure perpendicularly affixed to the lens; and a head having a diameter greater than the diameter of the retention structure and affixed to the second end of the retention structure.

The blade on the surgical tool can be sharp enough to penetrate the cornea.

The size of the blade and intraocular portions of the surgical tool will all fit inside the anterior chamber of the eye to be treated.

The secondary lens will be able to pass through the angle or bend in the surgical tool, as in some embodiments herein.

The angle or bend in the surgical tool can be measured from the beginning of the conduit at the operative end and the end of the conduit of the surgical end. The angle may be between 30 and 120 degrees, but may closer to 60 to 110 degrees. The radius of curvature may be very smooth and not abrupt.

Retention structure and protrusion can be used interchangeably when referring to the intraocular lens. Insertion point, insertion hole, and hole, can in some embodiments be used interchangeably when referring to the primary IOL. IOL being an intraocular lens.

It is, therefore, apparent that there has been provided, in accordance with the various objects of the present invention, an apparatus and method for the treatment of presbyopia in a pseudophakic eye, refractive error in a pseudophakic eye, and cataract in a cataractous eye. While the various objects of the present invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the present invention and the various embodiments described and envisioned by this specification, claims and the attached drawings.

What is claimed is:

1. An apparatus for treating a pseudophakic eye comprising a surgical tool having an operative head with a first edge and a conduit for placing a secondary intraocular lens in piggyback fashion into the pseudophakic eye.

2. The apparatus of claim 1 wherein the pseudophakic eye is being treated for presbyopia.

3. The apparatus of claim 1 wherein the pseudophakic eye is being treated for a refractive error.

4. The apparatus of claim 1 wherein the conduit is a delivery conduit for delivery of a secondary intraocular lens.

5. The apparatus of claim 1 further comprising an irrigation conduit.

6. The apparatus of claim 1 wherein the operative head further comprises a sharp tip.

7. The apparatus of claim 6 wherein the sharp tip is retractable

8. The apparatus of claim 1 further comprising a delivery conduit disposed within said surgical tool for delivery of a device with an energy source.

9. The apparatus of claim 1 further comprising a secondary intraocular lens contained within the delivery conduit.

10. The apparatus of claim 9, further comprising a guiding structure attached to the secondary intraocular lens that is contained within the delivery conduit.

11. The apparatus of claim 8 wherein the energy source is selected from the group consisting of heat, laser, and diathermy.

12. The surgical device of claim 1 adapted to be used to enter the eye through the peripheral cornea of the eye and deliver a secondary intraocular lens into the eye.

13. The apparatus of claim 9 wherein the secondary intraocular lens is used to treat a condition in a pseudophakic eye wherein the condition is selected from the group consisting of wrong power primary intraocular lens, myopia, hyperopia, astigmatism and higher order aberrations.

14. The apparatus of claim 9 wherein the secondary intraocular lens is selected from the group consisting of toric, single power, custom, and multi-focal.

15. The apparatus of claim 9 wherein the secondary intraocular lens comprises an expandable hydrogel.

16. The apparatus of claim 9 wherein the secondary intraocular lens is deformable to facilitate delivery of the secondary intraocular lens.

17. The apparatus of claim 9 wherein the secondary intraocular lens has structure to allow it to be placed through an insertion hole in a primary intraocular lens.

18. A secondary intraocular lens comprising a generally round lens affixed to a retention structure wherein the retention structure protrudes from the secondary intraocular lens and the retention structure fits into an eyelet on a primary intraocular lens.

19. The secondary intraocular lens of claim 18, further comprising a guiding structure attached to the secondary intraocular lens.

20. A blade on the surgical tool of claim 1 wherein the blade is used to penetrate the cornea of the eye and then retracted a distance of between 1 millimeter and 10 millimeters while other parts of the surgical tool remain disposed inside the eye to assist in the completion of the surgical procedure.
